(19) United States
(12) Patent Application Publication
(10) Pub. No.: US 2021/0315820 A1
(43) Pub. Date: Oct. 14, 2021

(54) LIPID NANOPARTICLE FORMULATIONS COMPRISING NUCLEIC ACID MIMICS

(71) Applicant: TRUCODE GENE REPAIR, INC., South San Francisco, CA (US)

(72) Inventors: Bo Ying, Arlington, MA (US); Derek Sim, Burlingame, CA (US)

(21) Appl. No.: 17/264,133

(22) PCT Filed: Jul. 25, 2019
(86) PCT No.: PCT/US2019/043382

§ 371 Date: Jan. 28, 2021

Related U.S. Application Data
(60) Provisional application No. 62/712,162, filed on Jul. 30, 2018.

(51) Int. Cl. A61K 9/127; A61K 9/51

(52) U.S. Cl. CPC A61K 9/1272; A61K 9/5123

(57) ABSTRACT

Described herein are lipid nanoparticles (LNPs), comprising neutral or positively charged nucleic acid mimics (NPNAMs) and optionally nucleic acids, and compositions thereof. Also described are methods of preparing LNPs comprising NPNAMs, and methods of use for intracellular gene editing. In particular, the LNPs comprising NPNAMs and optionally nucleic acids may be used in methods for the correction and/or treatment of a genetic disorder, disease, or condition in a subject.

### BACKGROUND

Intracellular gene editing generally requires a cellular delivery system that can penetrate a cellular membrane including in some cases the nuclear membrane, protect the gene editing payload under physiological conditions until delivered to a cell, and finally release the payload so that it is accessible to the cellular target nucleic acid. Cellular delivery systems such as polymeric nanoparticles (Gene Therapy, 20:658-669 (2013)), cationic liposomes (Hamilton, S. E. et al. Chem. Biol., 6:343-351 (1999)), and cell-penetrating peptide conjugates (CPPs, Pooga, M. Nature Biotechnol. 16:857-861 (1998)) have been all been employed to deliver nucleic acid mimics. However, effective cellular uptake of gene editing compositions remains a significant challenge, and there is a need in the art for new cellular delivery systems.

### SUMMARY

The present disclosure features a lipid nanoparticle (LNP) comprising a nucleic acid mimic (e.g., a neutral or positively charged nucleic acid mimic (NPNAM), e.g., a PNA oligomer, e.g., a tail-clamp PNA oligomer (tcPNA)), as well as compositions and related methods. An LNP disclosed herein may be used in methods to prevent or treat a disorder or condition, such as a genetic disorder, in a subject. The LNPs may be used to deliver NPNAMs and or other components, e.g., nucleic acids, into a cell both in vivo and in vitro.

In one aspect, the present disclosure features an LNP comprising: a) one or more or all of: (i) an ionizable lipid; (ii) a phospholipid; (iii) a sterol (e.g., cholesterol); and (iv) an alkylene glycol-containing lipid (a PEG-containing lipid); and b) a neutral or positively charged nucleic acid mimic (NPNAM). In an embodiment, the NPNAM comprises a PNA oligomer. In an embodiment, the PNA oligomer comprises a tail-clamp PNA oligomer (tcPNA). In an embodiment, the PNA oligomer comprises a gamma-substituted PNA subunit. In an embodiment, the gamma-substituted PNA subunit comprises a polyethylene glycol moiety at the gamma position. The PNA oligomer may comprises a PNA subunit having a structure of Formula (I), Formula (I-a), or Formula (I-b), described herein. In an embodiment, the amount of a PNA oligomer encapsulated and/or entrapped within the nanoparticle is between 0.1% to 50% (e.g., 0.1% to 25%, 1% to 10%, or 2% to 5%) by weight of PNA oligomers to the total weight of the LNP.

An LNP described herein may further comprise a load component, e.g., encapsulated and/or entrapped within the LNP. In an embodiment, the load component comprises a nucleic acid (e.g., a DNA, e.g., single-stranded DNA). In an embodiment, the nucleic acid comprises DNA. The load component may comprise any of the features disclosed herein.

The LNP of the present disclosure may comprise at least one, at least two, at least three, or all of an ionizable lipid, a phospholipid, a sterol, and a PEG-containing lipid. In an embodiment, the LNP comprises one or more or all of: (i) an ionizable lipid at a concentration between about 1 mol % to about 95 mol % (e.g. about 20 mol % to about 80 mol %); (ii) a phospholipid at a concentration between 0.1 mol % to about 50 mol % (e.g. between about 2.5 mol % to about 20 mol %); (iii) a sterol at a concentration between about 1 mol % to about 95 mol % (e.g. about 20 mol % to about 80 mol %); and (iv) a PEG-containing lipid at a concentration between about 0.1 mol % to about 50 mol % (e.g. between about 2.5 mol % to about 20 mol %).

In an embodiment, the LNP comprises one or more or all of the following properties: (i) the amount of PNA oligomer encapsulated and/or entrapped within the LNP is greater than or equal to 2 percent (2%) by weight of PNA oligomer to the total weight of the LNP; (ii) the diameter of the LNP is between 30 to 200 nanometers; or (iii) the LNP further comprises a load component (e.g., a nucleic acid), e.g., wherein the amount of the load component encapsulated and/or entrapped within the LNP is greater than or equal to 0.5 percent (0.5%) by weight of load component to the total weight of the LNP.

An LNP may be prepared by any method known in the art, for example, a method described herein.

In a second aspect, the present disclosure features a preparation comprising a plurality of LNPs, wherein each LNP of the plurality comprises: a) one or more or all of: (i) an ionizable lipid; (ii) a phospholipid; (iii) a sterol (e.g., cholesterol); and (iv) an alkylene glycol-containing lipid; and b) a neutral or positively charged nucleic acid mimic (NPNAM). In an embodiment, the NPNAM comprises a PNA oligomer. In an embodiment, the PNA oligomer comprises a tail-clamp PNA oligomer (tcPNA). In an embodiment, the PNA oligomer comprises a gamma-substituted PNA subunit. In an embodiment, the gamma-substituted PNA subunit comprises a polyethylene glycol moiety at the gamma position. The PNA oligomer may comprises a PNA subunit having a structure of Formula (I), Formula (I-a), or Formula (I-b), described herein. In an embodiment, the amount of a PNA oligomer encapsulated and/or entrapped within the plurality of LNPs is between 0.1% to 50% (e.g., 1% to 25%, 1% to 10%, or 2% to 5%) by weight of PNA oligomers to the total weight of the LNPs in the plurality.

A preparation comprising a plurality of LNPs described herein may further comprise a load component, e.g., encapsulated and/or entrapped within the plurality of LNPs. In an embodiment, the load component comprises a nucleic acid (e.g., a DNA, e.g., single-stranded DNA). In an embodiment, the nucleic acid comprises DNA. The load component may comprise any of the features disclosed herein.

The present disclosure further provides for methods for making an LNP or a preparation comprising a plurality of LNPs described herein, as well as methods of altering a target nucleic acid, methods of editing a target gene, methods of evaluating the extent of gene editing in a sample or subject, and methods for evaluating a sample or subject and determining a course of action responsive to the evaluation. The details of one or more embodiments of the disclosure are set forth herein. Other features, objects, and advantages of the invention will be apparent from the Detailed Description, the Figures, the Examples, and the Claims.

### BRIEF DESCRIPTION OF DRAWINGS

FIGS. 1A-1B are images of a generic peptide nucleic acid (PNA) subunit (FIG. 1A) and a generic tail-clamp peptide nucleic acid (tcPNA) (FIG. 1B). In FIG. 1A, B represents a nucleobase, R is a substituent on the PNA backbone, and α, β, and γ represent optionally substituted positions on the PNA backbone.

FIG. 2 is a schematic showing an exemplary apparatus and the process for generating PNA/DNA lipid nanoparticles (LNPs).

FIG. 3 is a is a chart depicting the size distribution of one exemplary batch of LNPs made using a method described herein (see, e.g., Example 2).

FIG. 4 is a graph showing the standard curve used to measure DNA concentration by Ribogreen.

FIG. 5 is a graph summarizing gene editing results for samples of a human B-cell line that is homozygous for the sickle cell mutation treated in vitro with LNPs containing varying ratios of an exemplary PNA (e.g., PNA-1) to donor DNA (1:1, 1:2, and 1:5, wt:wt) versus control studies (see, e.g., Example 4).

FIGS. 6A-6C are graphs summarizing gene editing results for samples of bone marrow cells (FIG. 6A), spleen cells (FIG. 6B), and liver cells (FIG. 6C) collected from sickle cell mice treated in vivo with LNPs containing varying ratios of an exemplary PNA (e.g., PNA-1) to donor DNA (1:1, 1:2, and 1:5, wt:wt) versus a control (untreated) group of mice. (see, e.g., Example 5).

### DETAILED DESCRIPTION

Disclosed herein is a lipid nanoparticle (LNP) that comprises a nucleic acid mimic (e.g., a neutral or positively charged nucleic acid mimic (NPNAM), e.g., a PNA oligomer, e.g., a tail-clamp PNA oligomer (tcPNA)), as well as compositions and methods related thereto, such as methods of making and using LNPs. In an embodiment, the LNP comprises an additional component, e.g., a load component (e.g., a nucleic acid). LNPs disclosed herein can be used in methods for the prevention and treatment of a disorder or condition, such as a genetic disorder, in a subject. The LNPs may be used to deliver NPNAMs and or other components, e.g., nucleic acids, into a cell both in vivo and in vitro. LNPs comprising a NPNAM and an optionally a nucleic acid may be capable of editing a gene, e.g., by binding to, acting as a primer for polymerase extension and/or replacing a target nucleic acid sequence (e.g., a DNA sequence) in a cell to modify the genome.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, the singular forms of words such as “a,” “an,” and “the,” include their corresponding plural references unless the context clearly dictates otherwise.

The terms “acquire” or “acquiring,” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a subject or measuring a value of a physical trait from a subject.

“Administer,” “administering,” or “administration,” as used herein, refer to providing or otherwise introducing an entity described herein (e.g., a LNP comprising a NPNAM), or a composition comprising said LNP, or providing the same to a subject.

“Deformulation,” as used herein, refers to breaking down a formulation (e.g., an LNP) in a manner that permits analysis of at least one of its active ingredients (e.g., a NPNAM or a nucleic acid).

“Effective amount” as used herein, refers to an amount of a LNP comprising a NPNAM, load component (e.g. nucleic acid), or mixture thereof, e.g., to treat or cure the phenotype of a disease, disorder, or condition. As will be appreciated by those of ordinary skill in this art, an effective amount may vary depending on such factors as the desired biological endpoint, the pharmacokinetics of the NPNAM and/or load component (e.g. nucleic acid), composition or LNP, the condition being treated, the mode of administration, and/or the age and health of the subject. An effective amount encompasses therapeutic and prophylactic treatment. For example, to treat sickle cell disease, an effective amount of LNP may be the amount needed to affect an in vivo or in vitro cell-based correction of a genetic defect causing sickle cell disease.

“NPNAM,” as used herein, refers to a neutral or positively charged nucleic acid mimic. For clarity, a NPNAM (as used herein) can comprise negatively charged groups or subunits so long as the net charge of the biopolymer is neutral or positive. In some embodiments, a NPNAM is a peptide nucleic acid (PNA) oligomer, e.g., a tail-clamp PNA. In some embodiments, a NPNAM is a PNA oligomer comprising the structure of PNA-1.

“Nucleic acid mimic” or “NAM,” as used herein, refers to a non-naturally occurring polymer composition that possesses the ability to sequence-specifically hybridize to a nucleic acid. Some non-limiting examples of nucleic acid mimics include peptide nucleic acids (PNAs, including all forms of PNAs as described in more detail herein), morpholinos (also known as phosphorodiamidate morpholino oligomers (PMOs), and morpholino oligomers (see: U.S. Pat. Nos. 5,142,047 and 5,185,444), pyrrolidine-amide oligonucleotide mimics (POMs; Samuel Tan, T. H. et al., Org. Biomol. Chem., 5:239-248 (2007), morpholinoglycine oligonucleotides (MGOs; Tatyana V. et al., Beilstein J. Org. Chem. 10:1151-1158 (2014)), and methyl phosphonates. In some embodiments, a NAM is a neutral or positively charged nucleic acid mimic (NPNAM).

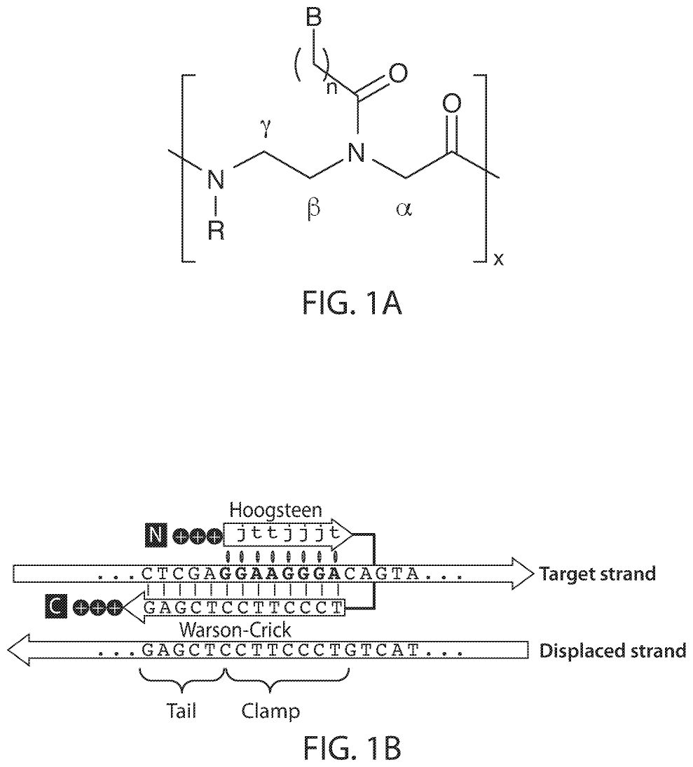

“Peptide nucleic acid,” “PNA,” or “PNA oligomer” as used herein, refer to a non-natural polymer composition comprising linked nucleobases capable of sequence specifically hybridizing to a nucleic acid. A PNA oligomer may comprise a nucleobase moiety and a backbone moiety, which can form hydrogen bonds with the nucleobase of a target nucleic acid. Exemplary PNAs are disclosed in or otherwise claimed in any of the following: U.S. Pat. Nos. 5,539,082, 5,527,675, 5,623,049, 5,714,331, 5,736,336, 5,773,571 or 5,786,461; (each of the foregoing are herein incorporated herein by reference in its entirety). The term “peptide nucleic acid” or “PNA” shall also apply to polymers comprising two or more subunits of kind described in the following publications: Diderichsen et al., Tetrahedron Lett. 37:475-478 (1996); Fujii et al., Bioorg. Med. Chem. Lett. 7:637-627 (1997); Jordan et al., Bioorg. Med. Chem. Lett. 7:687-690 (1997); Krotz et al., Tetrahedron Lett. 36:6941-6944 (1995); Lagriffoul et al., Bioorg. Med. Chem. Lett. 4:1081-1082 (1994); Lowe et al., J. Chem. Soc. Perkin Trans. 1, (1997) 1:539-546; Lowe et al., J. Chem. Soc. Perkin Trans. 1, 1:547-554 (1997); Lowe et al., J. Chem. Soc. Perkin Trans. 11:555-560 (1997); Petersen et al., Bioorg. Med. Chem. Lett. 6:793-796 (1996); Diederichsen, U., Bioorg. Med. Chem. Lett., 8:165-168 (1998); Cantin et al., Tetrahedron Lett., 38:4211-4214 (1997); Ciapetti et al., Tetrahedron, 53:1167-1176 (1997); Lagriffoule et al., Chem. Eur. J., 3:912-919 (1997); WIPO patent application WO96/04000 by Shah et al. and entitled “Peptide-based nucleic acid mimics (PENAMs)”; phosphono-PNA analogues (pPNAs) as described in: van der Laan, A. C. et al., Tetrahedron Let. 37:7857-7860 (1996); trans-4-hydroxy-L-proline nucleic acids (HypNAs) as described in Efimov et al., Nucleic Acids Res. 34(8):2247-2257 (2006); and (1S,2R/1R,2S)-cis-cyclopentyl PNAs (cpPNAs) as described in Govindaraju, T. et al., J. Org. Chem. 69(17):5725-34 (2004); each of the foregoing is herein incorporated herein by reference in its entirety. A PNA subunit of an exemplary PNA oligomer is depicted in FIG. 1A.

A “PNA subunit,” as used herein, refers to a PNA subunit within a PNA oligomer.

“Subject,” as used herein, refers to a human or non-human animal. In an embodiment, the subject is a human (i.e., a male or female, e.g., of any age group, a pediatric subject (e.g., infant, child, adolescent) or adult subject (e.g., young adult, middle-aged adult, or senior adult)). In an embodiment, the subject is a non-human animal, for example, a mammal (e.g., a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In an embodiment, the subject is a rodent (e.g., a mouse, a Townes sickle cell mouse, or a rat). In certain embodiments, the animal is a mammal. The animal may be a male or female and at any stage of development. A non-human animal may be a transgenic animal.

“Tail-clamp PNA oligomer” or “tcPNA”, as used herein, refers to a PNA oligomer capable of forming a PNA/DNA/PNA triplex upon binding to a target nucleic acid sequence (e.g., a target double stranded DNA sequence). A tcPNA comprises: i) a first region comprising a plurality of PNA subunits that participate in binding to the Hoogsteen face of a target nucleic acid sequence and ii) a second region comprising a plurality of PNA subunits that participate in binding to the Watson-Crick face of a target nucleic acid sequence. In an embodiment, the first region and second region of PNA subunits are linked by a linker (e.g., a polyethylene glycol linker). A tcPNA may further comprise iii) a third region comprising a plurality of PNA subunits that participate in binding to the Watson-Crick face of a target tail nucleic acid sequence and/or iv) a positively charged region comprising a plurality of positively charged moieties (e.g., positively charged amino acids) which may be present on a terminus of the tcPNA. An exemplary tcPNA is depicted in FIG. 1B.

“Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not. In some embodiments, treatment comprises curing a subject of a disease, e.g., or at least cure of the physical manifestation of the disease (e.g. cure of the phenotype), by, for example, effecting a genetic change to a sufficient number of cells of a subject.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 48 carbon atoms (“C₁-C₄₈ alkyl”). In some embodiments, an alkyl group has 1 to 36 carbon atoms (“C₁-C₃₆ alkyl”). In some embodiments, an alkyl group has 1 to 24 carbon atoms (“C₁-C₂₄ alkyl”). In some embodiments, an alkyl group has 1 to 18 carbon atoms (“C₁-C₁₈ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆alkyl”). Examples of C₁-C₂₄ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), octyl (C₈), nonyl (C₉), decyl (C₁₀), undecyl (C₁₁), dodecyl (or lauryl) (C₁₂), tridecyl (C₁₃), tetradecyl (or myristyl) (C₁₄), pentadecyl (C₁₅), hexadecyl (or cetyl) (C₁₆), heptadecyl (C₁₇), octadecyl (or stearyl) (C₁₈), nonadecyl (C₁₉), eicosyl (or arachidyl) (C₂₀), henicosanyl (C₂₁), docosanyl (C₂₂), tricosanyl (C₂₃), and tetracosanyl (C₂₄). Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 48 carbon atoms, one or more carbon-carbon double bonds, and no triple bonds (“C₂-C₄₈ alkenyl”). In some embodiments, an alkenyl group has 2 to 36 carbon atoms (“C₂-C₃₆ alkenyl”). In some embodiments, an alkenyl group has 2 to 24 carbon atoms (“C₂-C₂₄ alkenyl”). In some embodiments, an alkenyl group has 2 to 18 carbon atoms (“C₂-C₁₈ alkenyl”). In some embodiments, an alkenyl group has 2 to 12 carbon atoms (“C₂-C₁₂ alkenyl”). In some embodiments, an alkenyl group has 2 to 8 carbon atoms (“C₂-C₈ alkyl”). In some embodiments, an alkenyl group has 2 to 7 carbon atoms (“C₂-C₇ alkyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). The one or more carbon double bonds can have cis or trans (or E or Z) geometry. Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₂₄ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), nonenyl (C₉), nonadienyl (C₉), decenyl (C₁₀), decadienyl (C₁₀), undecenyl (C₁₁), undecadienyl (C₁₁), dodecenyl (C₁₂), dodecadienyl (C₁₂), tridecenyl (C₁₃), tridecadienyl (C₁₃), tetradecenyl (C₁₄), tetradecadienyl (e.g., myristoleyl) (C₁₄), pentadecenyl (C₁₅), pentadecadienyl (C₁₅), hexadecenyl (e.g., palmitoleyl) (C₁₆), hexadecadienyl (C₁₆), heptadecenyl (C₁₇), heptadecadienyl (C₁₇), octadecenyl (e.g., oleyl) (C₁₈), octadecadienyl, (e.g., linoleyl) (C₁₈), nonadecenyl (C₁₉), nonadecadienyl (C₁₉), eicosenyl (C₂₀), eicosadienyl (C₂₀), eicosatrienyl (C₂₀), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl.

As used herein, the term “alkynyl” refers to a radical of a straight-chain or branched hydrocarbon group having from 2 to 10 carbon atoms, one or more carbon-carbon triple bonds (“C₂-C₂₄ alkenyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “heteroalkyl” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. The term “alkenylene,” by itself or as part of another substituent, means, unless otherwise stated, a divalent radical derived from an alkene. An alkylene, alkenylene, alkynylene, or heteroalkylene group may be described as, e.g., a C₁-C₆-membered alkylene, C₁-C₆-membered alkenylene, C₁-C₆-membered alkynylene, or C₁-C₆-membered heteroalkylene, wherein the term “membered” refers to the non-hydrogen atoms within the moiety. In the case of heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—. Each instance of an alkylene, alkenylene, alkynylene, or heteroalkylene group may be independently optionally substituted, i.e., unsubstituted (an “unsubstituted alkylene”) or substituted (a “substituted heteroalkylene) with one or more substituents.

As used herein, “amino” refers to the radical —N(R¹⁰)(R¹¹), wherein each of R¹⁰ and R¹¹ is independently hydrogen, alkyl, alkenyl, alkynyl, aryl, heteroaryl, cycloalkyl, or heterocyclyl.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “cycloalkyl” refers to a radical of a non-aromatic cyclic hydrocarbon group having from 3 to 7 ring carbon atoms (“C₃-C₇ cycloalkyl”) and zero heteroatoms in the non-aromatic ring system. In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 7 ring carbon atoms (“C₅-C₇ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₇ cycloalkyl groups include, without limitation, the aforementioned C₃-C₆ cycloalkyl groups as well as cycloheptyl (C₇), cycloheptenyl (C₇), cycloheptadienyl (C₇), and cycloheptatrienyl (C₇), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

As used herein, the term “halo” refers to a fluorine, chlorine, bromine, or iodine radical (i.e., —F, —Cl, —Br, and —I).

As used herein, the term “heteroaryl,” refers to an aromatic heterocycle that comprises 1, 2, 3 or 4 heteroatoms selected, independently of the others, from nitrogen, sulfur and oxygen. As used herein, the term “heteroaryl” refers to a group that may be substituted or unsubstituted. A heteroaryl may be fused to one or two rings, such as a cycloalkyl, an aryl, or a heteroaryl ring. The point of attachment of a heteroaryl to a molecule may be on the heteroaryl, cycloalkyl, heterocycloalkyl or aryl ring, and the heteroaryl group may be attached through carbon or a heteroatom. Examples of heteroaryl groups include imidazolyl, furyl, pyrrolyl, thienyl, thiazolyl, isoxazolyl, isothiazolyl, thiadiazolyl, oxadiazolyl, pyridinyl, pyrimidyl, pyrazinyl, pyridazinyl, quinolyl, isoquinolinyl, indazolyl, benzoxazolyl, benzisooxazolyl, benzofuryl, benzothiazolyl, indolizinyl, imidazopyridinyl, pyrazolyl, triazolyl, oxazolyl, tetrazolyl, benzimidazolyl, benzoisothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidyl, pyrazolo[3,4]pyrimidyl or benzo(b)thienyl, each of which can be optionally substituted.

As used herein, the term “hydroxy” refers to the radical —OH.

As used herein, the term “oxo” refers to the radical —C═O.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. In some embodiments, ‘substantially free’, refers to: (i) an aliquot of an “R” form compound that contains less than 2% “S” form; or (ii) an aliquot of an “S” form compound that contains less than 2% “R” form. The term “enantiomerically pure” or “pure enantiomer” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure “R” form compound can comprise, for example, about 90% excipient and about 10% enantiomerically pure “R” form compound. In certain embodiments, the enantiomerically pure “R” form compound in such compositions can, for example, comprise, at least about 95% by weight “R” form compound and at most about 5% by weight “S” form compound, by total weight of the compound. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the compound. In certain embodiments, the active ingredient can be formulated with little or no excipient or carrier.

The symbol “ [symbol] ” as used herein in reference to a PNA oligomer, refers to a moiety at the terminus of the PNA oligomer or the attachment point to another region or atom within the PNA oligomer. In one embodiment, “ [symbol] ” refers to the N-terminus or the C-terminus of the PNA oligomer. In another embodiment, “ [symbol] ” refers to an attachment point to another PNA subunit or other region within a PNA oligomer. For example, in a tcPNA, “ [symbol] ” may refer to an attachment point to a linker (e.g., a polyethylene glycol linker) or a positively charged region comprising a plurality of positively charged moieties (e.g., positively charged amino acids).

### Lipid Nanoparticles

Described herein are lipid nanoparticles (LNPs) comprising a nucleic acid mimic (e.g., a NPNAM, e.g., a PNA oligomer) and methods of making and using the same. An LNP refers to a particle that comprises a lipid and a nucleic acid mimic, for example, an NPNAM. An LNP may further comprise a plurality of lipids, for example, at least one or more of an ionizable lipid, phospholipid, a sterol, or an alkylene glycol-containing lipid (e.g., a PEG-containing lipid), as well as a load component (e.g., a nucleic acid).

a. Lipids

The present disclosure features an LNP comprising a nucleic acid mimic (e.g., an NPNAM) and a lipid. Exemplary lipids include ionizable lipids, phospholipids, sterol lipids, alkylene glycol lipids (e.g., polyethylene glycol lipids), sphingolipids, glycerolipids, glycerophospholipids, prenol lipids, saccharolipids, fatty acids, and polyketides. In some embodiments, the LNP comprises a single type of lipid. In some embodiments, the LNP comprises a plurality of lipids. An LNP may comprise one or more of an ionizable lipid, a phospholipid, a sterol, or an alkylene glycol lipid (e.g., a polyethylene glycol lipid).

In an embodiment, the LNP comprises an ionizable lipid. An ionizable lipid is a lipid that comprises an ionizable moiety capable of bearing a charge (e.g., a positive charge or a negative charge) under certain conditions (e.g., at a certain pH range, e.g., under physiological conditions). An ionizable moiety may comprise an amine, carboxylic acid, hydroxyl, phenol, phosphate, sulfonyl, thiol, or a combination thereof. An ionizable lipid may be a cationic lipid or an anionic lipid. In additional to an ionizable moiety, an ionizable lipid may contain an alkyl or alkenyl group, e.g., greater than six carbon atoms in length (e.g., greater than about 8 carbons, 10 carbons, 12 carbons, 14 carbons, 16 carbons, 18 carbons, 20 carbons or more in length). Exemplary ionizable lipids include dilinoleylmethyl-4-dimethylaminobutyrate (DLin-MC3-DMA), 2,2-dilinoleyl-4-dimethylamino-[1,3]-dioxolane (DLin-K-DMA), 2,2-dilinoleyl-4-(2-dimethylaminoethyl)-[1,3]-dioxolane (DLin-KC2-DMA), 2,2-dilinoleyl-4-N-chloromethyl-N,N-dimethylamino-[1,3]-dioxolane (DLin-KC2-CIMDMA), 2,2-dilinoleyl-4-(3-dimethylaminopropyl)-[1,3]-dioxolane (DLin-KC3-DMA), 2,2-dilinoleyl-4-(4-dimethylaminobutyl)-[1,3]-dioxolane (DLin-KC4-DMA), 1,2-dilinoleyloxy-3-dimethylaminopropane (D-Lin-DMA), 1,2-dilinolenyloxy-dimethyl-3-aminopropane (D-Len-DMA), 1,2-dilinoleoyl-3-dimethylaminopropane (D-Lin-DAP), 1,2-dioleyloxy-dimethylaminopropane (DODMA), 1,2-distearyloxy-dimethyl-3-aminopropane (DSDMA), dioleoyl dimethyl-ammonium propane (DODAP), 1,2-dimyristyloxy-propyl-3-dimethyl-hydroxy ethyl ammonium bromide (DMRIE), dimethyl-[2-(sperminecarboxamido)ethyl]-2,3-bis(dioleyloxy)-1-propaniminium (DOSPA), 98N12-5, and C12-200. In some embodiments, the ionizable lipid comprises DLin-MC3-DMA, DLin-KC2-DMA, D-LinK-DMA, D-Lin-DAP, 98N12-5, C12-200, or DODMA. Additional ionizable lipids that may be included in an LNP described herein are disclosed in Jayaraman et al. (Angew. Chem. Int. Ed. 51:8529-8533 (2012)), Semple et al. (Nature Biotechnol. 28:172-176 (2010)), and U.S. Pat. Nos. 8,710,200 and 8,754,062, each of which is incorporated herein by reference in its entirety.

In some embodiments, an LNP comprises an ionizable lipid having a structure of Formula (II):

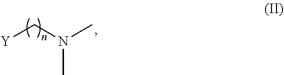

or a pharmaceutically acceptable salt thereof, wherein Y is

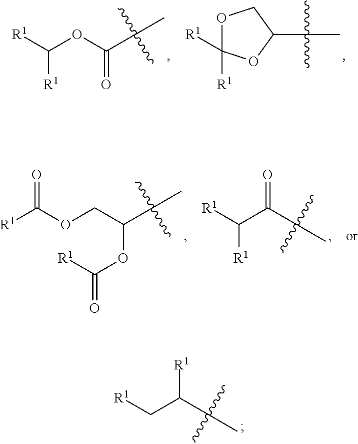

each R¹ is independently alkyl, alkenyl, alkynyl, or heteroalkyl, each of which is optionally substituted with R^{A}; each R^{A} is independently alkyl, halo, hydroxy, amino, cycloalkyl, or heterocyclyl; and n is an integer between 1 and 6.

In some embodiments, Y is

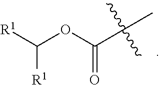

In some embodiments, each R¹ is independently alkyl (e.g., C₂-C₃₂ alkyl, C₄-C₂₈ alkyl, C₈-C₂₄ alkyl, C₁₂-C₂₂ alkyl, or C₁₆-C₂₀ alkyl). In some embodiments, each R¹ is independently alkenyl (e.g., C₂-C₃₂ alkenyl, C₄-C₂₈ alkenyl, C₈-C₂₄ alkenyl, C₁₂-C₂₂ alkenyl, or C₁₆-C₂₀ alkenyl). In some embodiments, each R¹ is independently C₁₆-C₂₀ alkenyl. In some embodiments, each R¹ is independently C₁₈ alkenyl. In some embodiments, each R¹ is independently linoleyl (or cis,cis-9,12-octadecadienyl). In some embodiments, each R¹ is the same. In some embodiments, each R¹ is different.

In some embodiments, n is an integer between 1 and 10, 1 and 8, 1 and 6, or 1 and 4. In some embodiments, n is 1, 2, 3, or 4. In some embodiments, n is 1, 2, or 3. In some embodiments, n is 1 or 2. In some embodiments, n is 1. In some embodiments, n is 2. In some embodiments, n is 3.

In some embodiments, the ionizable lipid is DLin-MC3-DMA. In some embodiments, the ionizable lipid is DLin-KC2-DMA. In some embodiments, the ionizable lipid is D-LinK-DMA. In some embodiments, the ionizable lipid is DLin-DMA. In some embodiments, the ionizable lipid is DLinDAP. In some embodiments, the ionizable lipid is 98N12-5. In some embodiments, the ionizable lipid is C12-200. In some embodiments, the ionizable lipid is DODMA.

An LNP may comprise an ionizable lipid at a concentration greater than about 0.1 mol %, e.g., of the total lipid composition of the LNP. In an embodiment, the LNP comprises an ionizable lipid at a concentration of greater than about 1 mol %, about 2 mol %, about 4 mol %, about 8 mol %, about 20 mol %, about 40 mol %, about 50 mol %, about 60 mol %, about 80 mol %, e.g., of the total lipid composition of the LNP. In an embodiment, the LNP comprises an ionizable lipid at a concentration of greater than about 20 mol %, about 40 mol %, or about 50 mol %. In an embodiment, the LNP comprises an ionizable lipid at a concentration between about 1 mol % to about 95 mol %, e.g., of the total lipid composition of the LNP. In an embodiment, the LNP comprises an ionizable lipid at a concentration between about 2 mol % to about 90 mol %, about 4 mol % to about 80 mol %, about 10 mol % to about 70 mol %, about 20 mol % to about 60 mol %, about 40 mol % to about 55 mol %, e.g., of the total lipid composition of the LNP. In an embodiment, the LNP comprises an ionizable lipid at a concentration between about 20 mol % to about 60 mol %. In an embodiment, the LNP comprises an ionizable lipid at a concentration between about 40 mol % to about 55 mol %.

In an embodiment, the LNP comprises a phospholipid. A phospholipid is a lipid that comprises a phosphate group and at least one alkyl, alkenyl, or heteroalkyl chain. A phospholipid may be naturally occurring or non-naturally occurring (e.g., a synthetic phospholipid). A phospholipid may comprise an amine, amide, ester, carboxyl, choline, hydroxyl, acetal, ether, carbohydrate, sterol, or a glycerol. In some embodiments, a phospholipid may comprise a phosphocholine, phosphosphingolipid, or a plasmalogen. Exemplary phospholipids include 1,2-dioleoyl-sn-glycero-3-phosphocholine (DOPC), 1,2-dipalmitoyl-sn-glycero-3-phosphocholine (DPPC), 1,2-dioleoyl-sn-glycero-3-phosphoethanolamine (DOPE), 1,2-distearoyl-sn-glycero-3-phosphocholine (DSPC), 1,2-dilauroyl-sn-glycero-3-phosphocholine (DLPC), 1,2-dimyristoyl-sn-glycero-3-phosphocholine (DMPC), 1,2-distearoyl-sn-glycero-3-phosphoethanolamine (DSPE), 1-myristoyl-2-oleoyl-sn-glycero-3-phosphocholine (MOPC), 1,2-diarachidonoyl-sn-glycero-3-phosphocholine (DAPC), 1-palmitoyl-2-linoleoyl-sn-glycero-3-phosphatidylcholine (PLPC), 1-palmitoyl-2-oleoyl-glycero-3-phosphocholine (POPC), 1-stearoyl-2-myristoyl-sn-glycero-3-phosphocholine (SMPC), 1-palmitoyl-2-myristoyl-sn-glycero-3-phosphocholine (PMPC), bis(monoacylglycerol)phosphate (BMP), L-α-phosphatidylcholine, 1,2-diheptadecanoyl-sn-glycero-3-phosphorylcholine (DHDPC), and 1-stearoyl-2-arachidonoyl-sn-glycero-3-phosphocholine (SAPC). Additional phospholipids that may be included in an LNP described herein are disclosed in Li, J. et al. (Asian J. Pharm. Sci. 10:81-98 (2015)), which is incorporated herein by reference in its entirety.

In some embodiments, an LNP comprises a phospholipid having a structure of Formula (III):

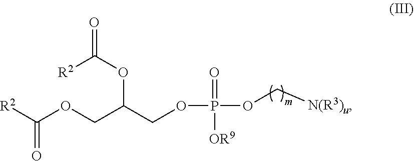

or a pharmaceutically acceptable salt thereof, wherein each R² is independently alkyl, alkenyl, or heteroalkyl; each R³ is independently hydrogen or alkyl; R⁹ is absent, hydrogen, or alkyl; each R^{B} is independently alkyl, halo, hydroxy, amino, cycloalkyl, or heterocyclyl; m is an integer between 1 and 4; and u is 2 or 3.

In some embodiments, each R² is independently alkyl (e.g., C₂-C₃₂ alkyl, C₄-C₂₈ alkyl, C₅-C₂₄ alkyl, C₁₂-C₂₂ alkyl, or C₁₆-C₂₀ alkyl). In some embodiments, each R² is independently alkenyl (e.g., C₂-C₃₂ alkyl, C₄-C₂₈ alkenyl, C₅-C₂₄ alkenyl, C₁₂-C₂₂ alkenyl, or C₁₆-C₂₀ alkenyl). In some embodiments, each R² is independently heteroalkyl (e.g., C₄-C₂₈ heteroalkyl, C₅-C₂₄ heteroalkyl, C₁₂-C₂₂ heteroalkyl, C₁₆-C₂₀ heteroalkyl). In some embodiments, each R² is independently C₁₆-C₂₀ alkyl. In some embodiments, each R² is independently C₁₇ alkyl. In some embodiments, each R² is independently heptadecyl. In some embodiments, each R² is the same. In some embodiments, each R² is different. In some embodiments, each R² is optionally substituted with R^{B}.

In some embodiments, one of R³ is hydrogen. In some embodiments, one of R³ is alkyl. In some embodiments, one of R³ is methyl. In some embodiments, each R³ is independently alkyl. In some embodiments, each R³ is independently methyl. In some embodiments, each R³ is independently methyl and u is 2. In some embodiments, each R³ is independently methyl and u is 3.

In some embodiments, R⁹ is absent. In some embodiments, R⁹ is hydrogen.

In some embodiments, m is an integer between 1 and 10, 1 and 8, 1 and 6, 1 and 4. In some embodiments, m is 1, 2, 3, or 4. In some embodiments, m is 1. In some embodiments, m is 2. In some embodiments, m is 3.

In some embodiments, the phospholipid is DSPC. In some embodiments, the phospholipid is DOPC. In some embodiments, the phospholipid is DPPC. In some embodiments, the phospholipid is DOPE.

An LNP may comprise a phospholipid at a concentration greater than about 0.1 mol %, e.g., of the total lipid composition of the LNP. In an embodiment, the LNP comprises a phospholipid at a concentration of greater than about 0.5 mol %, about 1 mol %, about 1.5 mol %, about 2 mol %, about 3 mol %, about 4 mol %, about 5 mol %, about 6 mol %, about 8 mol %, about 10 mol %, about 12 mol %, about 15 mol %, about 20 mol %, about 50 mol %, e.g., of the total lipid composition of the LNP. In an embodiment, the LNP comprises a phospholipid at a concentration of greater than about 1 mol %, about 5 mol %, or about 10 mol %. In an embodiment, the LNP comprises a phospholipid at a concentration between about 0.1 mol % to about 50 mol %, e.g., of the total lipid composition of the LNP. In an embodiment, the LNP comprises a phospholipid at a concentration between about 0.5 mol % to about 40 mol %, about 1 mol % to about 30 mol %, about 5 mol % to about 25 mol %, about 10 mol % to about 20 mol %, about 10 mol % to about 15 mol %, or about 15 mol % to about 20 mol %, e.g., of the total lipid composition of the LNP. In an embodiment, the LNP comprises a phospholipid at a concentration between about 5 mol % to about 25 mol %. In an embodiment, the LNP comprises a phospholipid at a concentration between about 10 mol % to 20 mol %.

In an embodiment, the LNP comprises a sterol. A sterol is a lipid that comprises a polycyclic structure and an optionally a hydroxyl or ether substituent, and may be naturally occurring or non-naturally occurring (e.g., a synthetic sterol). Sterols may comprise no double bonds, a single double bond, or multiple double bonds. Sterols may further comprise an alkyl, alkenyl, halo, ester, ketone, hydroxyl, amine, polyether, carbohydrate, or cyclic moiety. An exemplary listing of sterols includes cholesterol, ICE cholesterol, cholesterol hemisuccinate, dehydroergosterol, ergosterol, campesterol, β-sitosterol, stigmasterol, lanosterol, dihydrolanosterol, desmosterol, brassicasterol, lathosterol, zymosterol, 7-dehydrodesmosterol, avenasterol, campestanol, lupeol, and cycloartenol. In an embodiment, the sterol comprises cholesterol, ICE cholesterol, dehydroergosterol, ergosterol, campesterol, β-sitosterol, stigmasterol, lanosterol, dihydrolanosterol, desmosterol, brassicasterol, lathosterol, zymosterol, 7-dehydrodesmosterol, avenasterol, campestanol, lupeol, and cycloartenol. In some embodiments, the sterol comprises cholesterol, dehydroergosterol, ergosterol, campesterol, (3-sitosterol, or stigmasterol. Additional sterols that may be included in an LNP described herein are disclosed in Fahy, E. et al. (J. Lipid. Res. 46:839-862 (2005)), which is incorporated herein by reference in its entirety.

In some embodiments, an LNP comprises a sterol having a structure of Formula (IV):

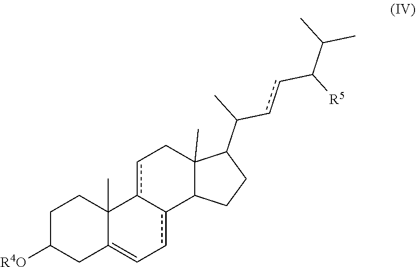

or a pharmaceutically acceptable salt thereof, wherein R⁴ is hydrogen, alkyl, heteroalkyl, or —C(O)R^{C}, R⁵ is hydrogen, alkyl, or —OR^{D}; each of R^{C} and R^{D} is independently hydrogen, alkyl, alkenyl, heteroalkyl, cycloalkyl, heterocyclyl, aryl or heteroaryl, wherein each alkyl, alkenyl, heteroalkyl, cycloalkyl, heterocyclyl, aryl or heteroaryl is optionally substituted with alkyl, halo, or oxo; and each “ [symbol] ” is either a single or double bond, and wherein each carbon atom participating in the single or double bond is bound to 0, 1, or 2 hydrogens, valency permitting.

In some embodiments, R⁴ is hydrogen. In some embodiments, R⁴ is alkyl (e.g., C₁-C₄ alkyl, C₄-C₈ alkyl, C₈-C₁₂ alkyl). In some embodiments, R⁴ is C(O)R^{C}, wherein R^{C} is alkyl (e.g., C₁-C₄ alkyl, C₄-C₈ alkyl, C₈-C₁₂ alkyl) or heteroaryl (e.g., a nitrogen-containing heteroaryl). In some embodiments, R⁴ is heteroalkyl (e.g., C₁-C₄ heteroalkyl, C₄-C₈ heteroalkyl, C₈-C₁₂ heteroalkyl). In some embodiments, R⁴ is heteroalkyl (e.g., C₁-C₄ heteroalkyl, C₄-C₈ heteroalkyl, C₈-C₁₂ heteroalkyl) substituted with oxo.

In some embodiments, R⁵ is hydrogen. In some embodiments, R⁵ is alkyl (e.g., C₁-C₄ alkyl, C₄-C₈ alkyl, C₈-C₁₂ alkyl).

In some embodiments, one of “ [symbol] ” is a single bond. In some embodiments, one of “ [symbol] ” is a double bond. In some embodiments, two of “ [symbol] ” are single bonds. In some embodiments, two of “ [symbol] ” are double bonds. In some embodiments, each “ [symbol] ” is a sing bond. In some embodiments, each “ [symbol] ” is a double bond.

In some embodiments, the sterol is cholesterol. In some embodiments, the sterol is cholesterol hemisuccinate. In some embodiments, the sterol is dehydroergosterol. In some embodiments, the sterol is ergosterol. In some embodiments, the sterol is campesterol. In some embodiments, the sterol is β-sitosterol. In some embodiments, the sterol is stigmasterol. In some embodiments, the sterol is a corticosteroid. (e.g., corticosterone, hydrocortisone, cortisone, or aldosterone).

An LNP may comprise a sterol at a concentration greater than about 0.1 mol %, e.g., of the total lipid composition of the LNP. In an embodiment, the LNP comprises a sterol at a concentration greater than about 0.5 mol %, about 1 mol %, about 5 mol %, about 10 mol %, about 15 mol %, about 20 mol %, about 25 mol %, about 35 mol %, about 40 mol %, about 45 mol %, about 50 mol %, about 55 mol %, about 60 mol %, about 65 mol %, or about 70 mol %, e.g., of the total lipid composition of the LNP. In an embodiment, the LNP comprises a sterol at a concentration greater than about 10 mol %, about 15 mol %, about 20 mol %, or about 25 mol %. In an embodiment, the LNP comprises a sterol at a concentration between about 1 mol % to about 95 mol %, e.g., of the total lipid composition of the LNP. In an embodiment, the LNP comprises a sterol at a concentration between about 5 mol % to about 90 mol %, about 10 mol % to about 85 mol %, about 20 mol % to about 80 mol %, about 20 mol % to about 60 mol %, about 20 mol % to about 50 mol %, or about 20 mol % to 40 mol %, e.g., of the total lipid composition of the LNP. In an embodiment, the LNP comprises a sterol at a concentration between about 20 mol % to about 50 mol %. In an embodiment, the LNP comprises a sterol at a concentration between about 30 mol % to about 60 mol %.

In some embodiments, the LNP comprises an alkylene glycol-containing lipid. An alkylene glycol-containing lipid is a lipid that comprises at least one alkylene glycol moiety, for example, a methylene glycol or an ethylene glycol moiety. In some embodiments, the alkylene glycol-containing lipid comprises a polyethylene glycol (PEG). An alkylene glycol-containing lipid may be a PEG-containing lipid. A PEG-containing lipid may further comprise an amine, amide, ester, carboxyl, phosphate, choline, hydroxyl, acetal, ether, heterocycle, or carbohydrate. PEG-containing lipids may comprise at least one alkyl or alkenyl group, e.g., greater than six carbon atoms in length (e.g., greater than about 8 carbons, 10 carbons, 12 carbons, 14 carbons, 16 carbons, 18 carbons, 20 carbons or more in length), e.g., in addition to a PEG moiety. In an embodiment, a PEG-containing lipid comprises a PEG moiety comprising at least 20 PEG monomers, e.g., at least 30 PEG monomers, 40 PEG monomers, 45 PEG monomers, 50 PEG monomers, 100 PEG monomers, 200 PEG monomers, 300 PEG monomers, 500 PEG monomers, 1000 PEG monomers, or 2000 PEG monomers. Exemplary PEG-containing lipids include 1,2-dimyristoyl-rac-glycero-3-methoxypolyethylene glycol (PEG-DMG, e.g., DMG-PEG2k), R-3-[(ω-methoxy poly(ethylene glycol)carbamoyl)]-1,2-dimyristyloxlpropyl-3-amine (PEG-c-DOMG), 1,2-distearoyl-rac-glycero-3-methylpolyoxyethylene (PEG-DSG), 1,2-dipalmitoyl-rac-glycero-3-methylpolyoxyethylene (PEG-DPG), 1,2-distearoyl-sn-glycero-3-phosphoethanolamine-N-[amino(polyethylene glycol)] (PEG-DSPE), N-(methylpolyoxyethylene oxycarbonyl)-1,2-dimyristoyl-sn-glycero-3-phosphoethanolamine (PEG-DMPE), N-(methylpolyoxyethylene oxycarbonyl)-1,2-dipalmitoyl-sn-glycero-3-phosphoethanolamine (PEG-DPPE), 1,2-dioleoyl-sn-glycero-3-phosphoethanolamine-N-[amino(polyethylene glycol (PEG-DOPE), and 2-dilauroyl-sn-glycero-3-phosphoethanolamine polyethylene glycol (PEG-DLPE). In some embodiments, the PEG-lipids include PEG-DMG (e.g., DMG-PEG2k), PEG-c-DOMG, PEG-DSG, and PEG-DPG. Additional PEG-lipids that may be included in an LNP described herein are disclosed in Fahy, E. et al. (J. Lipid. Res. 46:839-862 (2005) which is incorporated herein by reference in its entirety.

In some embodiments, an LNP comprises an alkylene glycol-containing lipid having a structure of Formula (V):

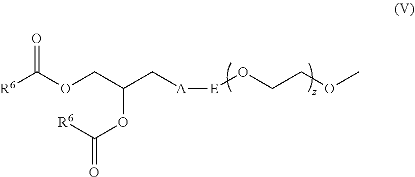

or a pharmaceutically acceptable salt thereof, wherein each R⁶ is independently alkyl, alkenyl, or heteroalkyl, each of which is optionally substituted with R^{E}; A is absent, O, CH₂, C(O), or NH; E is absent, alkyl, or heteroalkyl, wherein alkyl or heteroalkyl is optionally substituted with oxo; each R^{E} is independently alkyl, halo, hydroxy, amino, cycloalkyl, or heterocyclyl; and z is an integer between 10 and 200.

In some embodiments, each R⁶ is independently alkyl. In some embodiments, each R⁶ is independently heteroalkyl. In some embodiments, each R⁶ is independently alkenyl.

In some embodiments, A is O or NH. In some embodiments, A is CH₂. In some embodiments, A is oxo. In some embodiments, A is absent.

In some embodiments, E is alkyl. In some embodiments, E is heteroalkyl. In some embodiments, both A and E are not absent. In some embodiments, A is absent. In some embodiments, E is absent. In some embodiments, either one of A or E is absent. In some embodiments, both A and E are independently absent.

In some embodiments, z is an integer between 10 and 200 (e.g., between 20 and 180, between 20 and 160, between 20 and 120, between 20 and 100, between 40 and 80, between 40 and 60, between 40 and 50. In some embodiments, z is 45.

In some embodiments, the PEG-lipid is PEG-DMG (e.g., DMG-PEG2k). In some embodiments, the PEG-lipid is PEG-c-DOMG. In some embodiments, the PEG-lipid is PEG-DSG. In some embodiments, the PEG-lipid is PEG-DPG.

An LNP may comprise an alkylene glycol-containing lipid at a concentration greater than about 0.1 mol %, e.g., of the total lipid composition of the LNP. In an embodiment, the LNP comprises an alkylene glycol-containing lipid at a concentration of greater than about 0.5 mol %, about 1 mol %, about 1.5 mol %, about 2 mol %, about 3 mol %, about 4 mol %, about 5 mol %, about 6 mol %, about 8 mol %, about 10 mol %, about 12 mol %, about 15 mol %, about 20 mol %, about 50 mol %, e.g., of the total lipid composition of the LNP. In an embodiment, the LNP comprises an alkylene glycol-containing lipid at a concentration of greater than about 1 mol %, about 4 mol %, or about 6 mol %. In an embodiment, the LNP comprises an alkylene glycol-containing lipid at a concentration between about 0.1 mol % to about 50 mol %, e.g., of the total lipid composition of the LNP. In an embodiment, the LNP comprises an alkylene glycol-containing lipid at a concentration between about 0.5 mol % to about 40 mol %, about 1 mol % to about 35 mol %, about 1.5 mol % to about 30 mol %, about 2 mol % to about 25 mol %, about 2.5 mol % to about 20%, about 3 mol % to about 15 mol %, about 3.5 mol % to about 10 mol %, or about 4 mol % to 9 mol %, e.g., of the total lipid composition of the LNP. In an embodiment, the LNP comprises an alkylene glycol-containing lipid at a concentration between about 3.5 mol % to about 10 mol %. In an embodiment, the LNP comprises an alkylene glycol-containing lipid at a concentration between about 4 mol % to 9 mol %.

In some embodiments, the LNP comprises at least two types of lipids. In an embodiment, the LNP comprises two of an ionizable lipid, a phospholipid, a sterol, and an alkylene glycol-containing lipid. In some embodiments, the LNP comprises at least three types of lipids. In an embodiment, the LNP comprises three of an ionizable lipid, a phospholipid, a sterol, and a alkylene glycol-containing lipid. In some embodiments, the LNP comprises at least four types of lipids. In an embodiment, the LNP comprises each of an ionizable lipid, a phospholipid, a sterol, and an alkylene glycol-containing lipid.

The LNP (e.g., as described herein) may comprise one or more of the following components: (i) an ionizable lipid at a concentration between about 1 mol % to about 95 mol % (e.g. about 20 mol % to about 80 mol %); (ii) a phospholipid at a concentration between 0.1 mol % to about 50 mol % (e.g. between about 2.5 mol % to about 20 mol %); (iii) a sterol at a concentration between about 1 mol % to about 95 mol % (e.g. about 20 mol % to about 80 mol %); and (iv) a PEG-containing lipid at a concentration between about 0.1 mol % to about 50 mol % (e.g. between about 2.5 mol % to about 20 mol %). In an embodiment, the LNP comprises one of (i)-(iv). In an embodiment, the LNP comprises two of (i)-(iv). In an embodiment, the LNP comprises three of (i)-(iv). In an embodiment, the LNP comprises each of (i)-(iv). In some embodiments, the LNP comprises (i) and (ii). In some embodiments, the LNP comprises (i) and (iii). In some embodiments, the LNP comprises (i) and (iv). In some embodiments, the LNP comprises (ii) and (iii). In some embodiments, the LNP comprises (ii) and (iv). In some embodiments, the LNP comprises (iii) and (iv). In some embodiments, the LNP comprises (i), (ii), and (iii). In some embodiments, the LNP comprises (i), (ii), and (iv). In some embodiments, the LNP comprises (ii), (iii), and (iv).

The LNP (e.g., as described herein) may comprise one or more of the following components: (i) DLin-MC3-DMA at a concentration between about 1 mol % to about 95 mol % (e.g. about 20 mol % to about 80 mol %); (ii) DSPC at a concentration between 0.1 mol % to about 50 mol % (e.g. between about 2.5 mol % to about 20 mol %); (iii) cholesterol at a concentration between about 1 mol % to about 95 mol % (e.g. about 20 mol % to about 80 mol %); and (iv) DMG-PEG2k at a concentration between about 0.1 mol % to about 50 mol % (e.g. between about 2.5 mol % to about 20 mol %). In an embodiment, the LNP comprises two of (i)-(iv). In an embodiment, the LNP comprises three of (i)-(iv). In an embodiment, the LNP comprises each of (i)-(iv). In some embodiments, the LNP comprises (i) and (ii). In some embodiments, the LNP comprises (i) and (iii). In some embodiments, the LNP comprises (i) and (iv). In some embodiments, the LNP comprises (ii) and (iii). In some embodiments, the LNP comprises (ii) and (iv). In some embodiments, the LNP comprises (iii) and (iv). In some embodiments, the LNP comprises (iii) and (iv). In some embodiments, the LNP comprises (i), (ii), and (iii). In some embodiments, the LNP comprises (i), (ii), and (iv). In some embodiments, the LNP comprises (ii), (iii), and (iv).

The LNP (e.g., as described herein) may comprise one or more of the following components: (i) DLin-DMA at a concentration between about 1 mol % to about 95 mol % (e.g. about 20 mol % to about 80 mol %); (ii) DSPC at a concentration between 0.1 mol % to about 50 mol % (e.g. between about 2.5 mol % to about 20 mol %); (iii) cholesterol at a concentration between about 1 mol % to about 95 mol % (e.g. about 20 mol % to about 80 mol %); and (iv) DMG-PEG2k at a concentration between about 0.1 mol % to about 50 mol % (e.g. between about 2.5 mol % to about 20 mol %). In an embodiment, the LNP comprises two of (i)-(iv). In an embodiment, the LNP comprises three of (i)-(iv). In an embodiment, the LNP comprises each of (i)-(iv). In some embodiments, the LNP comprises (i) and (ii). In some embodiments, the LNP comprises (i) and (iii). In some embodiments, the LNP comprises (i) and (iv). In some embodiments, the LNP comprises (ii) and (iii). In some embodiments, the LNP comprises (ii) and (iv). In some embodiments, the LNP comprises (iii) and (iv). In some embodiments, the LNP comprises (iii) and (iv). In some embodiments, the LNP comprises (i), (ii), and (iii). In some embodiments, the LNP comprises (i), (ii), and (iv). In some embodiments, the LNP comprises (ii), (iii), and (iv).

The LNP (e.g., as described herein) may comprise one or more of the following components: (i) C12-200 at a concentration between about 1 mol % to about 95 mol % (e.g. about 20 mol % to about 80 mol %); (ii) DSPC at a concentration between 0.1 mol % to about 50 mol % (e.g. between about 2.5 mol % to about 20 mol %); (iii) cholesterol at a concentration between about 1 mol % to about 95 mol % (e.g. about 20 mol % to about 80 mol %); and (iv) DMG-PEG2k at a concentration between about 0.1 mol % to about 50 mol % (e.g. between about 2.5 mol % to about 20 mol %). In an embodiment, the LNP comprises two of (i)-(iv). In an embodiment, the LNP comprises three of (i)-(iv). In an embodiment, the LNP comprises each of (i)-(iv). In some embodiments, the LNP comprises (i) and (ii). In some embodiments, the LNP comprises (i) and (iii). In some embodiments, the LNP comprises (i) and (iv). In some embodiments, the LNP comprises (ii) and (iii). In some embodiments, the LNP comprises (ii) and (iv). In some embodiments, the LNP comprises (iii) and (iv). In some embodiments, the LNP comprises (iii) and (iv). In some embodiments, the LNP comprises (i), (ii), and (iii). In some embodiments, the LNP comprises (i), (ii), and (iv). In some embodiments, the LNP comprises (ii), (iii), and (iv).

The LNP (e.g., as described herein) may comprise one or more of the following components: (i) DLin-DMA at a concentration between about 1 mol % to about 95 mol % (e.g. about 20 mol % to about 80 mol %); (ii) DSPC at a concentration between 0.1 mol % to about 50 mol % (e.g. between about 2.5 mol % to about 20 mol %); (iii) cholesterol hemisuccinate at a concentration between about 1 mol % to about 95 mol % (e.g. about 20 mol % to about 80 mol %); and (iv) DMG-PEG2k at a concentration between about 0.1 mol % to about 50 mol % (e.g. between about 2.5 mol % to about 20 mol %). In an embodiment, the LNP comprises two of (i)-(iv). In an embodiment, the LNP comprises three of (i)-(iv). In an embodiment, the LNP comprises each of (i)-(iv). In some embodiments, the LNP comprises (i) and (ii). In some embodiments, the LNP comprises (i) and (iii). In some embodiments, the LNP comprises (i) and (iv). In some embodiments, the LNP comprises (ii) and (iii). In some embodiments, the LNP comprises (ii) and (iv). In some embodiments, the LNP comprises (iii) and (iv). In some embodiments, the LNP comprises (iii) and (iv). In some embodiments, the LNP comprises (i), (ii), and (iii). In some embodiments, the LNP comprises (i), (ii), and (iv). In some embodiments, the LNP comprises (ii), (iii), and (iv).

In an embodiment, the LNP comprises a ratio of ionizable lipid to phospholipid of about 50:1 to about 1:1 (e.g., 40:1, 32:3, 6:1, 7:1, 5:1, 24:5, 26:5, 10:3, 15:2, 16:7, 18:1, 3:1, 3:2, or 1:1). In an embodiment, the LNP comprises a ratio of ionizable lipid to phospholipid of about 15:2. In an embodiment, the LNP comprises a ratio of ionizable lipid to phospholipid of about 5:1. In an embodiment, the LNP comprises a ratio of ionizable lipid to a sterol of about 10:1 to about 1:10 (e.g., 9:1, 8:1, 8:7, 7:1, 7:5, 7:3, 6:1, 6:5, 5:1, 5:3, 4:1, 4:3, 3:1, 2:1, 1:1, 1:2, 1:3, 3:4, 1:4, 3:5, 1:5, 4:5, 1:6, 5:6, 7:6, 7:8, or 8:9). In an embodiment, the LNP comprises a ratio of ionizable lipid to an alkylene-containing lipid of about 1:10 to about 10:1 (e.g., 1:9, 1:8, 7:8, 7:1, 7:5, 7:3, 6:1, 6:5, 5:1, 5:3, 4:1, 4:3, 3:1, 2:1, 1:1, 1:2, 1:3, 3:4, 1:4, 3:5, 1:5, 4:5, 1:6, 5:6, 7:6, 7:8, or 8:9). In an embodiment, the LNP comprises a ratio of phospholipid to a alkylene-containing lipid of about 10:1 to about 1:10 (e.g., 9:1, 8:1, 8:7, 7:1, 7:5, 7:3, 6:1, 6:5, 5:1, 5:3, 4:1, 4:3, 3:1, 2:1, 1:1, 1:2, 1:3, 3:4, 1:4, 3:5, 1:5, 4:5, 1:6, 5:6, 7:6, 7:8, or 8:9). In an embodiment, the LNP comprises a ratio of a sterol to an alkylene-containing lipid of about 50:1 to about 1:1 (e.g., 40:1, 32:3, 6:1, 7:1, 5:1, 24:1, 22:1, 20:1, 22:5, 24:5, 26:5, 10:3, 15:2, 16:7, 18:1, 3:1, 3:2, or 1:1).

An LNP (e.g., described herein) comprises two of an ionizable lipid, a phospholipid, a sterol, and an alkylene glycol-containing lipid (e.g., PEG-containing lipid). An LNP (e.g., described herein) comprises three of an ionizable lipid, a phospholipid, a sterol, and an alkylene glycol-containing lipid (e.g., PEG-containing lipid). An LNP (e.g., described herein) comprises each of an ionizable lipid, a phospholipid, a sterol, and an alkylene glycol-containing lipid (e.g., PEG-containing lipid).

b. Nucleic Acid Mimics and NPNAMs

An LNP described herein comprises a nucleic acid mimic, for example, a neutral or positively charged nucleic acid mimic (NPNAM), as wells as related preparations and methods of making and using the same. In an embodiment, the NPNAM comprises a peptide nucleic acid (PNA) oligomer, morpholino, pyrrolidine-amide oligonucleotide mimic, morpholinoglycine oligonucleotide or methyl phosphonate.

In some embodiments, the NPNAM is a peptide nucleic acid (PNA). In some embodiments, the PNA oligomer is a tail-clamp peptide nucleic acid (tcPNA). A tcPNA may comprise: i) a first region comprising a plurality of PNA subunits that participate in binding to the Hoogsteen face of a target nucleic acid and ii) a second region comprising a plurality of PNA subunits that participate in binding to the Watson-Crick face of a target nucleic acid, wherein the first region and second region are covalently linked through a linker (e.g., a polyethylene-glycol linker). The tcPNA may further comprise: iii) a third region comprising a plurality of PNA subunits that participate in binding to the Watson-Crick face of a target tail nucleic acid sequence and iv) a positively charged region comprising positively charged amino acids (e.g., lysine residues) on at least one terminus of the tcPNA. In some embodiments, the tcPNA comprises one or more PNA subunits comprising a substituent at the gamma-position. In some embodiments, the tcPNA comprises one or more PNA subunits comprising a mini-PEG moiety at the gamma-position.

In some embodiments, the NPNAM is a PNA oligomer comprising a PNA subunit of Formula (I):

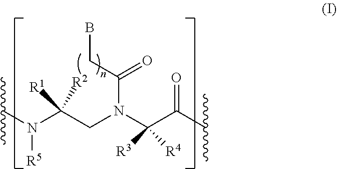

wherein B is a nucleobase; each of R¹, R², R³, and R⁴ is independently hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, and heteroaryl is optionally substituted with one or more R⁶; R⁵ is hydrogen or alkyl; each R⁶ is independently alkyl, heteroalkyl, amino, halo, oxo, or hydroxy; n is an integer between 1 and 10; and each “ [symbol] ” is independently the N-terminus of the PNA oligomer, the C-terminus of the PNA oligomer, or an attachment point to another PNA subunit.

In some embodiments, B is a naturally occurring nucleobase (e.g., adenine, cytosine, guanine, thymine, or uracil). In some embodiments, B is a non-naturally occurring nucleobase, e.g., pseudoisocytosine (i.e., J), 2,6-diaminopurine, 2-thiouracil, 7-deazaadenine, 7-deazaguanine, 2-thiopseudoisocytosine, 2-thiothymine, 2-thiocytosine, 5-chlorouracil, 5-bromouracil, 5-iodouracil, 5-chlorocytosine, 5-bromocytosine, 5-iodocytosine, 5-propynyl uracil, 5-propynyl cytosine, 6-azo uracil, 6-azo cytosine, 6-azo thymine, 7-methylguanine, 7-methyladenine, 8-azaguanine, 8-azaadenine, -deaza-2-aminoadenine (7-deaza-diaminopurine), 3-deazaguanine, 3-deazaadenine, 7-deaza-8-aza guanine, 7-deaza-8-aza adenine, 5-propynyl uracil and 2-thio-5-propynyl uracil, and tautomers thereof. In some embodiments, B is selected from adenine, guanine, thymine, cytosine, uracil, pseudoisocytosine, 2-thiopseudoisocytosine, 5-methylcytosine, 5-hydroxymethyl cytosine, xanthine, hypoxanthine, 2,6-diaminopurine, 2-thiouracil, 2-thiothymine, 2-thiocytosine, 5-chlorouracil, 5-bromouracil, 5-iodouracil, 5-chlorocytosine, 5-bromocytosine, 5-iodocytosine, 5-propynyl uracil, 5-propynyl cytosine, 6-azo uracil, 6-azo cytosine, 6-azo thymine, 7-methylguanine, 7-methyladenine, 8-azaguanine, 8-azaadenine, 7-deazaguanine, 7-deazaadenine, 7-deaza-2-aminoadenine, 3-deazaguanine, 3-deazaadenine, 7-deaza-8-aza guanine, 7-deaza-8-aza adenine, 5-propynyl uracil, 2-thio-5-propynyl uracil, and tautomers thereof. In some embodiments, B is selected from adenine, cytosine, guanine, thymine, uracil, pseudoisocytosine, 2,6-diaminopurine, 2-thiouracil, 7-deazaadenine, 7-deazaguanine, and tautomers thereof.

In some embodiments, each of R¹ and R² is independently heteroalkyl. In some embodiments, R¹ is heteroalkyl. In some embodiments, R¹ is heteroalkyl and R² is hydrogen. In some embodiments, R² is heteroalkyl. In some embodiments, R² is heteroalkyl and R¹ is hydrogen.

In some embodiments, each of R¹ and R² independently comprises a polyethylene glycol, e.g., a C2-C30 polyethylene glycol. In some embodiments, R¹ comprises a polyethylene glycol, e.g., a C2-C30 polyethylene glycol. In some embodiments, R¹ comprises a polyethylene glycol, e.g., a C2-C30 polyethylene glycol, and R² is hydrogen. In some embodiments, R² comprises a polyethylene glycol, e.g., a C2-C30 polyethylene glycol. In some embodiments, R² comprises a polyethylene glycol, e.g., a C2-C30 polyethylene glycol, and R¹ is hydrogen.

In some embodiments, each of R¹ and R² is independently heteroalkyl, wherein the heteroalkyl comprises the structure of Formula (VI-a) or (VI-b):

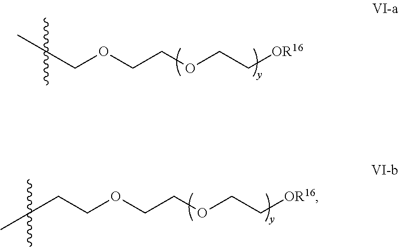

wherein R¹⁶ is hydrogen or alkyl (e.g., C1-C4 alkyl), y is an integer between 1 and 10, and “ [symbol] ” is carbon atom to which R¹ and R² are attached. In some embodiments, R¹ is Formula (VI-a), R¹⁶ is hydrogen or methyl (e.g., hydrogen), and y is 1. In some embodiments, R¹ is Formula (VI-a), R¹⁶ is hydrogen or methyl (e.g., hydrogen), y is 1, and R² is hydrogen. In some embodiments, R² is Formula (VI-a), R¹⁶ is hydrogen or methyl (e.g., hydrogen), y is 1, and R¹ is hydrogen.

In some embodiments, each of R³, R⁴, and R⁵ is independently hydrogen. In some embodiments, each of R³ and R⁴ is independently hydrogen. In some embodiments, R⁵ is hydrogen. In some embodiments, R³ is hydrogen. In some embodiments, R⁴ is hydrogen.

In some embodiments, n is 1. In some embodiments, n is 2. In some embodiments, n is 3.

In some embodiments of Formula (I), B is selected from adenine, cytosine, guanine, thymine, uracil, pseudoisocytosine, 2,6-diaminopurine, 2-thiouracil, 7-deazaadenine, and 7-deazaguanine; R¹ is a polyethylene glycol (e.g., a C2-C30 polyethylene glycol); each of R², R³, R⁴, and R⁵ is independently hydrogen; and n is 1.

In some embodiments of Formula (I), B is selected from adenine, cytosine, guanine, thymine, uracil, pseudoisocytosine, 2,6-diaminopurine, 2-thiouracil, 7-deazaadenine, and 7-deazaguanine; R¹ is —CH₂O—[CH₂CH₂O]ₑ—R₇ wherein e is 0, 1, 2, 3 or 4; R₇ is hydrogen, methyl, ethyl or t-butyl; each of R², R³, R⁴, and R⁵ is independently hydrogen; and n is 1.

In some embodiments of Formula (I), B is selected from adenine, cytosine, guanine, thymine, uracil, pseudoisocytosine, 2,6-diaminopurine, 2-thiouracil, 7-deazaadenine, and 7-deazaguanine; R² is —CH₂O—[CH₂CH₂O]ₑ—R₇ wherein e is 0, 1, 2, 3 or 4; R₇ is hydrogen, methyl, ethyl or t-butyl; each of R¹, R³, R⁴, and R⁵ is independently hydrogen; and n is 1.

In some embodiments of Formula (I), B is selected from adenine, cytosine, guanine, thymine, uracil, pseudoisocytosine, 2,6-diaminopurine, 2-thiouracil, 7-deazaadenine, and 7-deazaguanine; R³ is a —CH₂O—[CH₂CH₂O]ₑ—R₇ wherein e is 0, 1, 2, 3 or 4 and R₇ is hydrogen, methyl, ethyl or t-butyl; each of R¹, R², R⁴, and R⁵ is independently hydrogen; and n is 1.

In some embodiments of Formula (I), B is selected from adenine, cytosine, guanine, thymine, uracil, pseudoisocytosine, 2,6-diaminopurine, 2-thiouracil, 7-deazaadenine, and 7-deazaguanine; R⁴ is —CH₂O—[CH₂CH₂O]ₑ—R₇ wherein e is 0, 1, 2, 3 or 4; R₇ is hydrogen, methyl, ethyl or t-butyl; each of R¹, R², R³, and R⁵ is independently hydrogen; and n is 1.

In some embodiments, the NPNAM is a PNA oligomer comprising greater than 2, 3, 4, 5, 6, 7, 8, 9, 10, 15, 20, 25, 30, 35, 40, 45, or 50 PNA subunits. In some embodiments, the NPNAM is a PNA oligomer comprising between 10 to 25 PNA subunits. In some embodiments, the NPNAM is a PNA oligomer comprising between 20 to 35 PNA subunits. In some embodiments, the NPNAM is a PNA oligomer comprising between about 2 to 50 PNA subunits, e.g., between about 4 and 45, 6 and 40, 8 and 35, 10 and 30, and 15 and 15 PNA subunits.

In some embodiments, the NPNAM is a PNA oligomer comprising a PNA monomer subunit of Formula (I-a):

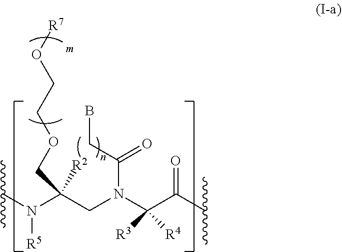

wherein B is a nucleobase; each of R², R³, and R⁴ is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, and heteroaryl is optionally substituted with one or more R⁶; R⁵ is hydrogen or alkyl; each R⁶ is independently alkyl, heteroalkyl, amino, halo, oxo, or hydroxy; R⁷ is hydrogen or alkyl; m is an integer between 0 and 10, and n is an integer between 1 and 10; and each “ [symbol] ” is independently the N-terminus of the PNA oligomer, the C-terminus of the PNA oligomer, or an attachment point to another PNA subunit.

In some embodiments, B is a naturally occurring nucleobase (e.g., adenine, cytosine, guanine, thymine, or uracil). In some embodiments, B is a non-naturally occurring nucleobase, e.g., pseudoisocytosine (i.e., j), 2,6-diaminopurine, 2-thiouracil, 7-deazaadenine, and 7-deazaguanine. In some embodiments, B is selected from adenine, cytosine, guanine, thymine, uracil, pseudoisocytosine, 2,6-diaminopurine, 2-thiouracil, 7-deazaadenine, and 7-deazaguanine.

In some embodiments, R² is heteroalkyl (e.g., a polyethylene glycol, e.g., a C2-C30 polyethylene glycol). In some embodiments, R² is hydrogen.

In some embodiments, each of R³, R⁴, and R⁵ is independently hydrogen. In some embodiments, each of R³ and R⁴ is independently hydrogen. In some embodiments, R⁵ is hydrogen. In some embodiments, R³ is hydrogen. In some embodiments, R⁴ is hydrogen.

In some embodiments, R⁷ is hydrogen. In some embodiments, R⁷ is alkyl (e.g., methyl, ethyl or t-butyl).

In some embodiments, m is 0. In some embodiments, m is 1. In some embodiments, m is 2. In some embodiments, m is 3. In some embodiments, n is 1. In some embodiments, n is 2. In some embodiments, n is 3. In some embodiments, m is 1 and n is 1. In some embodiments, m is 2 and n is 1. In some embodiments, m is 3 and n is 1.

In some embodiments, B is selected from adenine, cytosine, guanine, thymine, uracil, pseudoisocytosine, 2,6-diaminopurine, 2-thiouracil, 7-deazaadenine, and 7-deazaguanine; each of R², R³, R⁴, R⁵, and R⁷ is independently hydrogen; m is 2 and n is 1.

In some embodiments, the NPNAM is a PNA oligomer comprising a PNA monomer subunit of Formula (I-b):

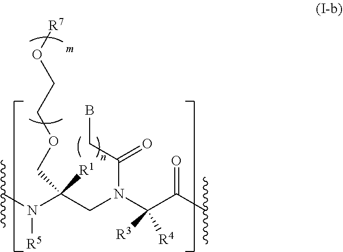

wherein B is a nucleobase; each of R¹, R³, and R⁴ is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, and heteroaryl is optionally substituted with one or more R⁶; R⁵ is hydrogen or alkyl; each R⁶ is independently alkyl, heteroalkyl, amino, halo, oxo, or hydroxy; R⁷ is hydrogen or alkyl; m is an integer between 0 and 10, and n is an integer between 1 and 10; and each “ [symbol] ” is independently the N-terminus of the PNA oligomer, the C-terminus of the PNA oligomer, or an attachment point to another PNA subunit.

In some embodiments, B is a naturally occurring nucleobase (e.g., adenine, cytosine, guanine, thymine, or uracil). In some embodiments, B is a non-naturally occurring nucleobase, e.g., pseudoisocytosine (i.e., j), 2,6-diaminopurine, 2-thiouracil, 7-deazaadenine, and 7-deazaguanine. In some embodiments, B is selected from adenine, cytosine, guanine, thymine, uracil, pseudoisocytosine, 2,6-diaminopurine, 2-thiouracil, 7-deazaadenine, and 7-deazaguanine.

In some embodiments, R¹ is heteroalkyl (e.g., a polyethylene glycol, e.g., a C2-C30 polyethylene glycol). In some embodiments, R¹ is hydrogen.

In some embodiments, each of R³, R⁴, and R⁵ is independently hydrogen. In some embodiments, each of R³ and R⁴ is independently hydrogen. In some embodiments, R⁵ is hydrogen. In some embodiments, R³ is hydrogen. In some embodiments, R⁴ is hydrogen.

In some embodiments, R⁷ is hydrogen. In some embodiments, R⁷ is alkyl (e.g., methyl, ethyl or t-butyl).

In some embodiments, m is 0. In some embodiments, m is 1. In some embodiments, m is 2. In some embodiments, m is 3. In some embodiments, n is 1. In some embodiments, n is 2. In some embodiments, n is 3. In some embodiments, m is 1 and n is 1. In some embodiments, m is 2 and n is 1. In some embodiments, m is 3 and n is 1.

In some embodiments, B is selected from adenine, cytosine, guanine, thymine, uracil, pseudoisocytosine, 2,6-diaminopurine, 2-thiouracil, 7-deazaadenine, and 7-deazaguanine; each of R², R³, R⁴, R⁵, and R⁷ is independently hydrogen; m is 2 and n is 1.

In some embodiments, the NPNAM comprising a PNA subunit of Formula (I) is a tail-clamp PNA oligomer (tcPNA). In some embodiments, the NPNAM comprising a PNA subunit of Formula (I-a) is a tail-clamp PNA oligomer (tcPNA). In some embodiments, the NPNAM comprising a PNA subunit of Formula (I-b) is a tail-clamp PNA oligomer (tcPNA).

In some embodiments, the NPNAM comprises a PNA oligomer having the sequence of PNA-1. In some embodiments, the NPNAM comprises a PNA oligomer having the sequence of PNA-2.

An LNP may comprise a single NPNAM or a plurality of NPNAMs. In some embodiments, an LNP comprises 1 NPNAM. In some embodiments, an LNP comprises a plurality of NPNAMs, for example, at least 2 NPNAMs, 3 NPNAMs, 4 NPNAMs, 5 NPNAMs, 6 NPNAMs, 7 NPNAMs, 8 NPNAMs, 9 NPNAMs, 10 NPNAMs, 15 NPNAMs, 20 NPNAMs, 25 NPNAMs, 30 NPNAMs, 40 NPNAMs, 50 NPNAMs, 60 NPNAMs, 70 NPNAMs, 80 NPNAMs, 90 NPNAMs, 100 NPNAMs, 150 NPNAMs, 200 NPNAMs, 300 NPNAMs, 400 NPNAMs, 500 NPNAMs, 600 NPNAMs, 700 NPNAMs, 800 NPNAMs, 900 NPNAMs, or 1,000 NPNAMs. In some embodiments, an LNP comprises 10-50 NPNAMs. In some embodiments, an LNP comprises 10-100 NPNAMs. In some embodiments, an LNP comprises between 100-1,000 NPNAMs. In some embodiments, an LNP comprises between 500-1,000 NPNAMs.

An LNP may comprise a single PNA oligomer or a plurality of PNA oligomers. In some embodiments, an LNP comprises 1 PNA oligomer. In some embodiments, an LNP comprises a plurality of PNA oligomers, for example, at least 2 PNAs, 3 PNAs, 4 PNAs, 5 PNAs, 6 PNAs, 7 PNAs, 8 PNAs, 9 PNAs, 10 PNAs, 15 PNAs, 20 PNAs, 25 PNAs, 30 PNAs, 40 PNAs, 50 PNAs, 60 PNAs, 70 PNAs, 80 PNAs, 90 PNAs, 100 PNAs, 150 PNAs, 200 PNAs, 300 PNAs, 400 PNAs, 500 PNAs, 600 PNAs, 700 PNAs, 800 PNAs, 900 PNAs, or 1,000 PNAs. In some embodiments, an LNP comprises 10-50 PNA oligomers. In some embodiments, an LNP comprises 10-100 PNA oligomers. In some embodiments, an LNP comprises between 100-1,000 PNA oligomers. In some embodiments, an LNP comprises between 500-1,000 PNA oligomers.

The amount of a NPNAM (e.g., a PNA oligomer) encapsulated and/or entrapped within the LNP may vary depending on the identity of the NPNAM (e.g., PNA oligomer) or plurality of NPNAMs (e.g., PNA oligomers). For example, the amount of NPNAM (e.g., PNA oligomer) may be between 0.05% and 50% by weight of NPNAMs (e.g., PNA oligomers) to the total weight of the LNP. In some embodiments, the amount of NPNAM in the LNP is greater than about 0.05%, e.g., greater than about 0.1%, 0.5%, 1%, 2%, 3%, 4%, 5%, 6%, 7%, 8%, 9%, 10%, 12.5%, 15%, 20%, 25%, 30%, 35%, 40%, 45%, or 50% by weight of NPNAM to the total weight of the LNP. In some embodiments, the amount of PNA oligomer in the LNP is greater than about 0.05%, e.g., greater than about 0.1%, 0.5%, 1%, 2%, 3%, 4%, 5%, 6%, 7%, 8%, 9%, 10%, 12.5%, 15%, 20%, 25%, 30%, 35%, 40%, 45%, or 50% by weight of PNA to the total weight of the LNP. In some embodiments the amount of PNA oligomer in the LNP is between 0.1% and 50% by weight of PNA oligomers to the total weight of the LNP, or between 1% and 25% by weight of PNA oligomers to the total weight of the LNP, or between 1% and 10% by weight of PNA oligomer to the total weight of the LNP, or between 2% to 5% by weight of PNA oligomer to the total weight of the LNP.

An LNP described herein may comprise a single type of NPNAM (e.g., a single type of PNA oligomer, or a PNA oligomer of a single sequence), or may comprise multiple types of NPNAMs. In some embodiments, the LNP comprises a single type of NPNAM. In some embodiments, the LNP comprise a plurality of types of NPNAMs (e.g., a plurality of PNA oligomers).

c. Load Components

In some embodiments, an LNP further comprises a load component. In some embodiments, the load component is an additional biological component (e.g., a polymeric biological component), for example, a nucleic acid or polypeptide. In some embodiments, the load component is a nucleic acid. In some embodiments, the nucleic acid is double stranded. In some embodiments, the nucleic acid is single stranded. In some embodiments, the load component is an oligonucleotide. In some embodiments, the load component is a single stranded DNA. In some embodiments, the load component is a single stranded RNA. In some embodiments, the load component is a double stranded DNA. In some embodiments, the load component is a double stranded RNA. In some embodiments, the load component is an mRNA. In some embodiments, the load component is an siRNA. In some embodiments, the load component is an antisense oligomer (PNA or DNA).

In some embodiments, the load component is a nucleic acid (e.g., DNA) between 5 and 250 nucleotides in length. In some embodiments, the load component is a nucleic acid (e.g., DNA) between 10 and 200 nucleotides in length. In some embodiments, the load component is a nucleic acid (e.g., DNA) between 20 and 100 nucleotides in length). In some embodiments, the load component is a nucleic acid (e.g., DNA) between 40 and 80 nucleotides in length. In some embodiments, the load component is a nucleic acid (e.g., DNA) between 60 and 70 nucleotides in length. In some embodiments, the load component is a nucleic acid (e.g., DNA) between 20 and 40 nucleotides in length. In some embodiments, the load component is a single stranded nucleic acid (e.g., DNA) between 20 and 70 nucleotides in length. In some embodiments, the load component is a double stranded nucleic acid (e.g., DNA) between 20 and 70 nucleotides in length.

In some embodiments, the load component is a nucleic acid and comprises one or more phosphorothioate linkages at a terminus (e.g., the 5′ terminus or the 3′ terminus). In some embodiments, the load component is a nucleic acid and comprises one or more phosphorothioate linkages at an internucleoside linkage. In some embodiments, the load component comprises more than one phosphorothioate linkages at each terminus, for example, at each of its 3′ and 5′ terminus. In some embodiments, the nucleic acid comprises 2, 3, 4, 5, 6, 7, 8, 9, 10 or more phosphorothioate linkages at a terminus or at an internucleoside linkage.

In some embodiments, the load component comprises a nucleic acid having a sequence which is the same or the complement of a sequence to which the NPNAM, e.g., a clamp system, e.g., a tail clamp system, e.g., a PNA oligomer comprising a sequence of PNA-1 as described herein, has Watson Crick homology. In some embodiments, a load component comprises a nucleic acid having a sequence which is the same or the complement of a sequence to which the NPNAM, e.g., a tcPNA, e.g., a PNA oligomer comprising a sequence of PNA-1 as described herein, has Hoogsteen homology. In some embodiments, a load component comprises a nucleic acid having a sequence which is the same or the complement of a sequence that is within 1,000, 500, or 200 base pairs of a sequence to which the NPNAM e.g., a tcPNA, e.g., a PNA oligomer comprising a sequence of PNA-1 as described herein, has Watson Crick homology. In some embodiments, the load component comprises a nucleic acid having a sequence which is the same or the complement of a sequence that is within 1,000, 500, or 200 base pairs of a sequence to which the NPNAM, e.g., a tcPNA, e.g., a PNA oligomer comprising a sequence of PNA-1 as described herein, has Hoogsteen homology.

In some embodiments, an LNP comprises an NPNAM and a load component. In some embodiments, the ratio of NPAM to load component is equal (ie. 1:1). In some embodiments, the ratio of NPAM to load component is greater than 1:1, for example, about 1:1.1, about 1:1.2, about 1:1.3, about 1:1.5, about 1:2, about 1:3, about 1:4, about 1:5, about 1:10, or about 1:25 NPNAM to load component. In some embodiments, the ratio of load component to NPNAM greater than 1:1, for example, about 1:1.1, about 1:1.2, about 1:1.3, about 1:1.5, about 1:2, about 1:3, about 1:4, about 1:5, or about 1:10 load component to NPNAM. In an embodiment, the ratio of NPNAM to load component is about 1:1. In an embodiment, the ratio of NPNAM to load component is about 1:2. In an embodiment, the ratio of NPNAM to load component is about 1:5.

d. Features of LNPs

An LNP described herein (e.g., comprising an NPNAM and a lipid, and optionally a load component) may have a certain ratio of components. For example, the LNP described herein may comprise a particular ratio of a lipid or a plurality of lipids to an NPNAM. In an embodiment, the ratio of a plurality of lipids to an NPNAM (e.g., a PNA oligomer) is between 100:1 to 1:100 (e.g. about 75:1 to 1:75, about 60:1 to 1:60, 100:1 to about 5:1, 80:1 to about 5:1, 60:1 to about 5:1, or about 50:1 to about 5:1). In some embodiments, the ratio of a plurality of lipids to an NPNAM (e.g., a PNA oligomer) is about 100:1, 95:1, 90:1, 85:1, 80:1, 75:1, 70:1, 65:1, 60:1, 55:1, 50:1, 45:1, 40:1, 35:1, 30:1, 28:1, 26:1, 24:1, 25:1, 22:1, 20:1, 18:1, 16:1, 14:1, 12:1, 10:1, 8:1, 6:1, 4:1, 2:1, 1:1, 1:2, 1:4, 1:6, 1:8, 1:10, 1:12, 1:14, 1:16, 1:18, 1:20, 1:22, 1:24, 1:25, 1:26, 1:28, 1:30, 1:35, 1:40, 1:45, 1:50, 1:55, 1:60, 1:65, 1:70, 1:75, 1:80, 1:85, 1:90, 1:95, or 1:100.

In some embodiments, an LNP described herein has a diameter between 5 and 500 nm, e.g., between 10 and 400 nm, 20 and 300 nm, 25 and 250 nm, 30 and 200 nm, and 30 and 100 nm. The diameter of an LNP may be determined by any method known in the art, for example, dynamic light scattering. In some embodiments, an LNP has a diameter between 50 and 100 nm, between 70 and 100 nm, and between 80 and 100 nm. In an embodiment, an LNP has a diameter of about 90 nm. In some embodiments, an LNP described herein has a diameter greater than about 30 nm. In some embodiments, an LNP has a diameter greater than about 35 nm, about 40 nm, about 45 nm, about 50 nm, about 60 nm, about 70 nm, about 80 nm, about 90 nm, about 100 nm, about 120 nm, about 140 nm, about 160 nm, about 180 nm, or about 200 nm. In an embodiment, an LNP has a diameter greater than about 70 nm. In an embodiment, an LNP has a diameter greater than about 90 nm.

In some embodiments, a plurality of LNPs described herein has an average diameter greater than about 30 nm. In some embodiments, a plurality of LNPs has an average diameter greater than about 35 nm, about 40 nm, about 45 nm, about 50 nm, about 60 nm, about 70 nm, about 80 nm, about 90 nm, about 100 nm, about 120 nm, about 140 nm, about 160 nm, about 180 nm, or about 200 nm. In an embodiment, a plurality of LNPs has an average diameter greater than about 70 nm.

### Methods of Making of Lipid Nanoparticles

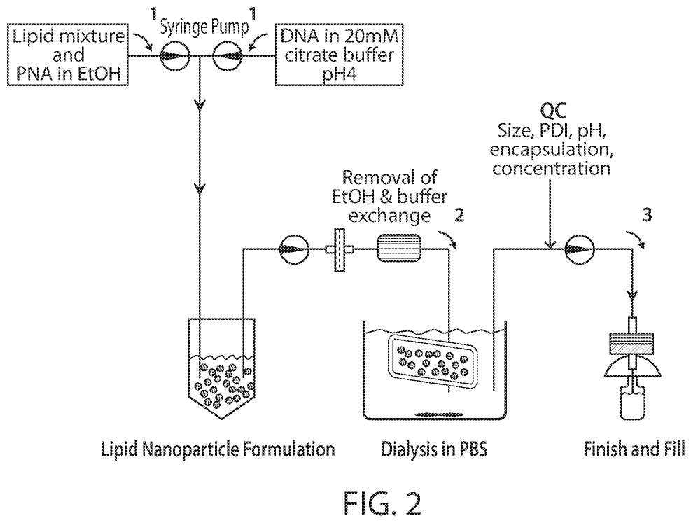

Described herein are methods for producing an LNP that comprises a lipid and a nucleic acid mimic (e.g., a NPNAM, e.g., a PNA oligomer). An example of the process described herein is depicted in FIG. 2. In some embodiments, two solutions are prepared and ultimately combined [Step 1]. In some embodiments, the first solution comprises a lipid or a plurality of lipids in a solvent. In some embodiments, the first solution further comprises a NPNAM (e.g., a PNA oligomer, e.g., a tcPNA oligomer) in a solvent. The solvent may be any water miscible solvent (e.g., ethanol, methanol, isopropanol, acetonitrile, dimethylformamide, dioxane, tetrahydrofuran). In some embodiments, the first solution comprises a small percentage of water. The first solution may comprise up to at least 60% by volume of at water, e.g., up to at least about 0.05%, 0.1%, 0.5%%, 1%, 2%, 3%, 4%, 5%, 10%, 15%, 20%, 25%, 30%, 35%, 40%, 45%, 50%, 55% or 60% by volume of water. In an embodiment, the first solution comprises between about 0.05% and 60% by volume water, e.g., between about 0.05% and 50%, about 0.05% and 40%, or about 5% and 20% by volume water.

The first solution may comprise a single type of NPNAM or a plurality of NPNAMs, e.g., of different NPNAM sequences. In an embodiment, the first solution comprises a single type of NPNAM (e.g., a PNA oligomer, e.g., a tcPNA). In an embodiment, the first solution comprises a plurality of NPNAMs (e.g., PNA oligomers, e.g., tcPNAs), wherein the NPNAMs comprise different sequences and bind to different target nucleic acid sequences.

In some embodiments, the first solution comprises a single type of lipid, for example, an ionizable lipid, a phospholipid, a sterol, or a PEG-containing lipid. In some embodiments, the first solution comprises a plurality of lipids. In some embodiments, the plurality comprises an ionizable lipid, a phospholipid, a sterol, or a PEG-containing lipid. In some embodiments, the plurality of lipids comprise cholesterol, 1,2-distearoyl-sn-glycero-3-phosphocholine (DSPC), 1,2-dimyristoyl-rac-glycero-3-methylpolyoxyethylene2000 (DMG-PEG2k), and dilinoleylmethyl-4-dimethylaminobutyrate (DLin-MC3-DMA). The plurality of lipids may exist in any ratio. In an embodiment, the plurality of lipids comprises an ionizable lipid, a phospholipid, a sterol, or a PEG-containing lipid of the above lipids in a particular ratio (e.g., a ratio described herein).

In some embodiments, the second solution is water. In some embodiments, the second solution is an aqueous buffer. The second solution may comprise a load component, e.g., a nucleic acid (e.g., a single-stranded DNA). In some embodiments, the nucleic acid is a DNA oligomer (e.g. a donor DNA). The second solution may comprise a small percentage of water miscible organic solvent. The second solution may comprise up to at least 60% by volume of at least one water miscible organic solvent, e.g., up to at least about 0.05%, 0.1%, 0.5%%, 1%, 2%, 3%, 4%, 5%, 10%, 15%, 20%, 25%, 30%, 35%, 40%, 45%, 50%, 55% or 60% by volume of at least one organic solvent (e.g., a water miscible organic solvent). In an embodiment, the second solution comprises between about 0.05% and 60% by volume organic solvent, e.g., between about 0.05% and 50%, about 0.05% and 40%, or about 5% and 20% by volume organic solvent (e.g., a water miscible organic solvent). The aqueous buffer solution may be an aqueous solution of citrate buffer. In some embodiments, the aqueous buffer solution is a citrate buffer solution with a pH between 4-6 (e.g., a pH of about 4, about 5, or about 6). In an embodiment, the aqueous buffer solution is a citrate buffer solution with a pH of about 6.

In some embodiments, the first solution is mixed with the second solution to form lipid nanoparticles (e.g., FIG. 2, step 1). The lipid nanoparticles may be formed through nanoprecipitation. The nanoprecipitation may thereby encapsulate or contain the NPNAM(s) within the LNP. The nanoprecipitation may also encapsulate or entrap the nucleic acid(s) in the LNP. In some embodiments, the suspension of lipid nanoparticle formulation is collected within a vessel.

In some embodiments, the suspension is subjected to buffer exchange and concentration (e.g., FIG. 2, step 2). The buffer exchange and concentration may comprise dialysis, e.g., in phosphate buffer solution (PBS). The dialysis of the LNP suspension may remove excess reagents, solvents, free NPNAM or free nucleic acid.

In some embodiments, the suspension may be passed through a filter of proper pore size so as to sterilize the LNPs. The filter may be of an appropriate pore size to remove microbes (e.g., 0.22 micrometer filter capable or removing bacteria and virus particles).

In some embodiments, the solution comprising a mixture of the first and second solutions comprising a suspension of LNPs can be diluted. In some embodiments, the pH of the solution comprising a mixture of the first and second solutions comprising a suspension of LNPs can be adjusted. Dilution or adjustment of the pH of the nanoparticle suspension may be achieved with the addition of water or aqueous buffer. In some embodiments, no dilution or adjustment of the pH of the nanoparticle suspension is carried out. In some embodiments, both dilution and adjustment of the pH of the nanoparticle suspension is carried out.

In some embodiments, excess reagents, solvents, free NPNAM or free nucleic acid may be removed from the suspension by tangential flow filtration (TFF) (e.g., diafiltration). The organic solvent (e.g., ethanol) and buffer may also be removed from the suspension with TFF. In some embodiments, the nanoparticle suspension is subjected to dialysis and not TFF. In some embodiments, the nanoparticle suspension is subjected to TFF and not dialysis. In some embodiments, the nanoparticle suspension is subjected to both dialysis and TFF.

In some embodiments, the solution comprising a mixture of the first and second solutions comprising a suspension of LNPs is diluted. In some embodiments, the pH of the solution comprising a mixture of the first and second solutions comprising a suspension of LNPs is adjusted. Dilution or adjustment of the pH of the nanoparticle suspension may be achieved with the addition of water or aqueous buffer. In some embodiments, no dilution or adjustment of the pH of the nanoparticle suspension is carried out. In some embodiments, both dilution and adjustment of the pH of the nanoparticle suspension is carried out.

In some embodiments, the above process is carried out in an apparatus. With reference to FIG. 2, the apparatus may comprise a first solvent supply and a second solvent supply. The first solvent supply may comprise at least one neutral or positively charged nucleic acid mimic (NPNAM) and at least one water miscible organic solvent. The first solvent supply may comprise a lipid mixture. In an embodiment, the first solvent supply comprises a mixture of NPNAMs and lipids in a water miscible organic solvent. In some embodiments, the first solvent supply further comprises up to at least 60% by volume of water, e.g., up to at least about 0.05%, 0.1%, 0.5%%, 1%, 2%, 3%, 4%, 5%, 10%, 15%, 20%, 25%, 30%, 35%, 40%, 45%, 50%, 55% or 60% by volume of water. In an embodiment, the first solvent supply comprises between about 0.05% and 60% by volume water, e.g., between about 0.05% and 50%, about 0.05% and 40%, or about 5% and 20% by volume water.

In some embodiments, the second solvent supply comprises water, an aqueous solution or aqueous buffer. The aqueous buffer solution may be an aqueous solution of citrate buffer. In an embodiment, the aqueous buffer solution is a citrate buffer solution. The second solvent supply may further comprise a load component, e.g., a nucleic acid or mixture of nucleic acids (e.g., DNA). In some embodiments, the nucleic acid is a donor DNA sequence. In some embodiments, the second solvent supply further comprises up to at least 60% by volume of at least one water miscible organic solvent, e.g., up to at least about 0.05%, 0.1%, 0.5%%, 1%, 2%, 3%, 4%, 5%, 10%, 15%, 20%, 25%, 30%, 35%, 40%, 45%, 50%, 55% or 60% by volume of at least one organic solvent (e.g., a water miscible organic solvent). In an embodiment, the second solvent supply comprises between about 0.05% and 60% by volume organic solvent, e.g., between about 0.05% and 50%, about 0.05% and 40%, or about 5% and 20% by volume organic solvent (e.g., a water miscible organic solvent).

In some embodiments the apparatus further comprises at least one junction to which the first solvent supply and second solvent supply are in fluid connection. This junction may permit efficient mixing of the first and second solvents (and their associated contents) as they are forced into said junction and as they exit the junction into a post-junction conduit that contains the post-junction fluid stream. In some cases, the junction is referred to as a ‘tee’ mixer or ‘tee’ or just “T” junction because it can be made to resemble the letter “T”. In some embodiments, the junction to permit efficient mixing of the first and second solvents comprises a “Y” type junction (e.g., a “Y” junction). In some embodiments, the junction to permit efficient mixing of the first and second solvents comprises a cross junction.

In some embodiments, a supply comprises any physical mode that can supply the first and second solvents to the junction (or the diluent supply to the post-junction conduit as described below). For example, each supply can be a reservoir wherein the components of each solvent to be supplied to the junction are first mixed in appropriate concentrations and then delivered to the appropriate junction. Alternatively, the ‘supply’ can be a conduit into which more than one solution is combined (typically at another junction) in a ratio suitable to produce the supply of reagent(s) in the proper ratios and concentrations needed to feed said junction. Simply stated, so long as the relevant junction is properly supplied, it is not relevant how such supply is created.

In some embodiments the apparatus comprises one or more pumps in fluid connection with junction and one or more of the supplies. In an embodiment, the apparatus comprises a pump that is in fluid connection with the first solvent supply and the junction and a pump that is in fluid connection with the second solvent supply (FIG. 2). In some embodiments, pressurized chambers or gravity are used to deliver the first solvent supply and the second solvent supply to the junction.

In some embodiments the apparatus comprises at least one additional diluent supply. In some embodiments, there is no additional diluent supply. The diluent supply may comprise water, or an aqueous solution (e.g., an aqueous buffer solution). The diluent supply may be connected to the post-junction conduit. In an embodiment, the diluent supply is connected to the post-junction conduit by a Tee junction, Y junction, or a cross junction. The fluid connection between the diluent supply and the junction may optionally comprise a pump that is suitable to pump the contents of the diluent supply through said junction and into the post-junction conduit. In some embodiments, gravity or a pressurized supply are used instead of a pump to provide flow into the post-junction conduit.

In some embodiments, the apparatus comprises a mixing vessel (FIG. 2). The mixing vessel may be positioned to capture the post-junction fluid stream that exits the post-junction conduit. The mixing vessel can be open or closed. The mixing vessel may additionally comprise at least one input port in fluid connection with the post-junction conduit. The pH of the fluid stream may also be adjusted within the mixing vessel. In an embodiment, the pH of the fluid stream is adjusted to a pH of about 8.

In some embodiments, the apparatus includes an exit port (FIG. 2). The exit port may be structured and positioned to permit fluid contents of the mixing vessel to exit the mixing vessel and flow into a post-mixing vessel conduit. The apparatus may also optionally comprise yet another pump structured and positioned to permit the pumping of a solution comprising LNPs that has accumulated in said mixing vessel into the post-mixing vessel conduit to thereby produce a post-mixing vessel fluid stream contained by said post-mixing vessel conduit.

In some embodiments, the apparatus includes a dialysis device. The dialysis device may be in fluid communication with the post-mixing vessel conduit (FIG. 2). The passing through the dialysis device may remove excess solvents, buffer, NPNAM(s), nucleic acid(s) and/or any small molecules. In some embodiments, the passing through the dialysis devices removes contaminates that are undesired in a pharmaceutical product. Dialysis devices may be affixed in-line in the post-mixing vessel conduit. Contaminates may be shunted away from the dialysis devices and post-mixing vessel conduit. Dialysis devices may be structured and positioned to route effluent from the dialysis devices into a post-dialysis conduit that contains a post-dialysis fluid stream. In some embodiments, the apparatus has a dialysis device and no TFF device. In some embodiments, the apparatus has a TFF device and no dialysis devices. In some embodiments, the apparatus has both a TFF device and a dialysis device.

In some embodiments, the apparatus includes a tangential flow filtration (TFF) device. The TFF devices may be in fluid communication with the post-mixing vessel conduit. The post-mixing vessel fluid stream may pass through the TFF devices. The passing through the TFF devices may remove excess solvents, buffer, NPNAM(s), nucleic acid(s) and/or any small molecules. In some embodiments, the passing through the TFF devices removes contaminates that are undesired in a pharmaceutical product. TFF devices may be affixed in-line in the post-mixing vessel conduit. Contaminates may be shunted away from the TFF devices and post-mixing vessel conduit. TFF devices may be structured and positioned to route effluent from the TFF devices into a post-TFF conduit that contains a post-TFF fluid stream.

In some embodiments, the apparatus also includes a buffer exchange vessel (FIG. 2). The buffer exchange vessel may capture the post-TFF fluid stream. The buffer exchange vessel may be open or closed. The buffer exchange vessel may comprise at least one input port in fluid connection with the post-TFF conduit to permit entry of the post-TFF fluid stream. The buffer exchange vessel may be structured and positioned to receive effluent from the post-TFF conduit. In some embodiments, the buffer exchange vessel permits buffer exchange of the LNPs suspension. The buffer exchange may involve adding or adjusting the suspension of LNPs to achieve a preferred concentration of excipients and other reagents and compositions prior to finish and fill of pharmaceutical product/ingredients. In some embodiments, the apparatus comprises a buffer exchange vessel conduit that permits flow of the contents of the drug product formulation vessel to a finish and fill apparatus. The apparatus may also optionally comprise yet another pump structured and positioned to permit the pumping of a solution comprising LNPs that has accumulated in said drug product formulation vessel.

In some embodiments, it is possible to reroute the flow of some of the LNP suspension from the apparatus. In some embodiments, the flow of the LNP suspension is rerouted from the post-drug product formulation vessel conduit. The flow of LNP suspension may be rerouted for any of the quality control (QC) purposes one may wish to perform. Non-limiting examples of QC processes include sizing of the LNPs, confirming pH of the solution carrying the LNPs, the pH of the LNPs, the zeta potential of the LNPs, the concentration of LNPs in the solution, the amount of API in the LNPs.

It is to be understood that not all (or even any) of the optional components of the apparatus must be present to operate. However, the apparatus is so configured to permit efficient preparation of LNPs formulated with encapsulated/entrapped NPNAM (and optionally one or more loading components (e.g. nucleic acids)) and provides for integrated removal of excess reagents as well as for post-production operations such as sterilization and finish and fill.

In one aspect, the present disclosure features a method comprising treating a sample of LNPs comprising NPNAMs and optionally nucleic acids, with a fluid comprising a detergent (e.g., Triton X-100) for a period of time suitable to degrade the lipid layer and thereby release the encapsulated and/or entrapped NPAMs and optionally nucleic acids. In an embodiment, the method further comprises analyzing the sample for the presence, absence, and/or amount of the released NPNAMs and optionally nucleic acids.

In some embodiments, the present disclosure features a method of manufacturing, or evaluating, a LNP or preparation of LNPs comprising providing a preparation of LNPs described herein, and acquiring, directly or indirectly, a value for a preparation parameter. In an embodiment, the method further comprises making the preparation of LNPs by a method described herein (e.g., the method illustrated by FIG. 2). In an embodiment, the method further comprises evaluating the value for the preparation parameter, e.g., by comparing it with a standard or reference value. In an embodiment, wherein responsive to the evaluation, the method further comprises selecting a course of action, and optionally, performing the action. For example, the method may comprise providing a preparation of LNPs comprising a NPNAM (e.g., a PNA) acquiring a value for a preparation parameter (e.g., average particle size), evaluating the preparation the value of the preparation parameter by comparing it with a standard or reference value, selecting a course of action (e.g., selecting to administer the preparation of LNPs to a subject), and performing the action (administering the preparation of LNPs to a subject).

In the presence of a target sequence, an LNP may lead to interaction of the target sequence with an NPNAM. For example, in some embodiments, an LNP, or the contents of the LNP, allows binding of its component NPNAM to a target nucleic acid sequence, e.g., as evaluated by UV melting temperature in hybridization experiments (e.g., at 260 nm), thermodynamic analysis, or surface plasmon resonance. In some embodiments, a LNP, or the contents of the LNP, when contacted with a target nucleic acid, decreases the Tm of a target nucleic acid sequence, e.g., as evaluated by UV melting temperature in hybridization experiments (e.g., at 260 nm), thermodynamic analysis, or surface plasmon resonance. In some embodiments, a LNP, or the contents of the LNP, when contacted with a target nucleic acid, promotes melting or dissociation of the strands of a target nucleic acid sequence, e.g., as evaluated by strand invasion assay. In some embodiments, a LNP, or the contents of the LNP, when contacted with a target nucleic acid, allows its component NPNAM to cleave a target nucleic acid sequence. In some embodiments, a LNP, or the contents of the LNP, when contacted with a target nucleic acid, allows its component NPNAM and nucleic acid to edit a target nucleic acid sequence, e.g., as evaluated by NGS or ddPCR.

In some embodiments, a LNP is prepared by a method described herein.

### Methods of Targeting a Gene

The present disclosure further entails methods of altering a target nucleic acid using the LNPs and related preparations described herein. In some embodiments, the present disclosure features a method of altering a target nucleic acid, comprising providing a preparation of LNPs described herein, e.g., comprising a NPNAM and/or described herein (e.g., a NPNAM of the sequence PNA-1, or a preparation of LNPs made by a method described herein, e.g., as depicted in FIG. 2). In some embodiments, the method of altering a target nucleic acid further comprises contacting the NPNAM of a LNP with a target nucleic acid under conditions sufficient to alter the target nucleic acid. In some embodiments, the method comprises administering an LNP or preparation of LNPs to a subject. In some embodiments, the method comprises administering an LNP or preparation of LNPs to a cell.

The method of altering a target nucleic acid may be performed in an in vitro cell system, in a cell, or in vivo (e.g., in a subject, e.g., a human subject). In some embodiments, the method is performed in an in vitro cell free system. In some embodiments, the method is performed in a cell. In some embodiments, the cell is a fertilized egg. The cell may be a cultured cell, e.g., a cell from a cell line, or may be a cell derived from a subject. In some embodiments, the method is performed in vivo, e.g., in a subject. In some embodiments, the subject is a human (i.e., a male or female, e.g., of any age group, a pediatric subject (e.g., infant, child, adolescent) or adult subject (e.g., young adult, middle-aged adult, or senior adult)). In an embodiment, the subject is a non-human animal, for example, a mammal (e.g., a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In an embodiment, the subject is a rodent (e.g., a mouse, a Townes sickle cell mouse, or a rat). In certain embodiments, the animal is a mammal. The animal may be a male or female and at any stage of development. A non-human animal may be a transgenic animal. In some embodiments, the subject is not yet born (e.g. in-utero). In some embodiments, the subject is a human fetus.

An LNP or a preparation of LNPs comprising an NPNAM (e.g., as described herein) may be capable of altering a nucleic acid. In some embodiments, the LNP or preparation of LNPs has one or more of the following properties:
a) it alters the state of association of a target nucleic acid. For example, the LNP or preparation of LNPs may alter the state of association of the two strands of a double-stranded nucleic acid;
b) it alters the helical structure of a target nucleic acid (e.g., a target double-stranded nucleic acid);
c) it alters the topology of a target nucleic acid (e.g., by introducing a kink or bend in a strand of the nucleic acid);
d) its association with a target nucleic acid is accompanied by or results in recruitment of a nucleic acid-modifying protein (e.g., enzyme), for example, a member of the nucleotide excision repair pathway, to a target double stranded nucleic acid. Exemplary members of the nucleotide excision repair pathway include XPA, RPA, XPF, and XPG, or a functional variant or fragment thereof;
e) it cleaves a strand of a target nucleic acid (e.g., a double-stranded nucleic acid); or
f) it alters the sequence of a target nucleic acid. In some embodiments, the sequence of a target nucleic acid is altered to the sequence of a template nucleic acid. In some embodiments, the sequence of a target nucleic acid is altered from a mutant or disorder-associated sequence (e.g., allele) to a non-mutant or non-disease associated sequence (e.g., allele).

In some embodiments, the LNP or preparation of LNPs comprises two of: (a)-(f). In some embodiments, the LNP or preparation of LNPs comprises three of: (a)-(f). In some embodiments, the LNP or preparation of LNPs comprises four of: (a)-(f). In some embodiments, the LNP or preparation of LNPs comprises five of: (a)-(f). In some embodiments, the LNP or preparation of LNPs comprises each of: (a)-(f). In some embodiments, the LNP or preparation of LNPs comprises (a). In some embodiments, the LNP or preparation of LNPs comprises (b). In some embodiments, the LNP or preparation of LNPs comprises (c). In some embodiments, the LNP or preparation of LNPs comprises (d). In some embodiments, the LNP or preparation of LNPs comprises (e). In some embodiments, the LNP or preparation of LNPs comprises (f).

An LNP or preparation of LNPs may comprise some or all of the components useful to alter a nucleic acid and/or to edit a gene. In some embodiments, the NPNAM (e.g., the PNA oligomer) of the LNP is packaged into a single composition of matter for delivery to the cell(s) or subject (e.g. all NPNAMs are loaded into a single LNP, or the NPNAMs are packaged in separate LNPs than the load component).

The LNP, or the contents of the LNP, may promote a particular effect in a target nucleic acid sequence. For example, an LNP, or the contents of the LNP, when contacted with a target nucleic acid may allow binding of its component NPNAM to a target nucleic acid sequence. In some embodiments, an LNP, or the contents of the LNP, when contacted with a target nucleic acid may provide a decrease in the melting point (Tm) of a target nucleic acid sequence (e.g., a decrease of about 0.5%, 1%, 5%, 10%, 20%, 30%, 40%, 50%, or more). In some embodiments, an LNP, or the contents of the LNP, when contacted with a target nucleic acid may promote melting or dissociation of the strands of a target nucleic acid sequence (e.g., a melting or dissociation of about 0.5%, 1%, 5%, 10%, 20%, 30%, 40%, 50%, or more of the strands of the target sequence). In some embodiments, an LNP, or the contents of the LNP, when contacted with a target nucleic acid, may allow its component NPNAM to cleave a target nucleic acid sequence (e.g., effect cleavage in about 0.5%, 1%, 5%, 10%, 20%, 30%, 40%, 50%, or more target nucleic acid sequences). In some embodiments, an LNP, or the contents of the LNP, when contacted with a target nucleic acid may allow its component NPNAM and nucleic acid to edit a target nucleic acid sequence (e.g., edit about 0.5%, 1%, 5%, 10%, 20%, 30%, 40%, 50%, or more of the strands of the target sequence).

The extent of gene editing achieved by an LNP, contents of the LNP, or a preparation of LNPs may be measured by any method known in the art. In some embodiments, the extent of gene editing achieved may be determined by polymerase chain reaction (PCR) analysis or a particular sequencing method. In an embodiment, the extent of gene editing achieved by an LNP or the contents of an LNP is determined with droplet digital PCR (ddPCR). In an embodiment, the extent of gene editing achieved by an LNP or the contents of an LNP is determined with next generation sequencing (NGS). In an embodiment, the extent of gene editing achieved by an LNP or the contents of an LNP is determined whole genome sequencing (WGS).

### Examples

In order that the invention described herein may be more fully understood, the following examples are set forth. The examples described in this application are offered to illustrate the LNPs and related methods provided herein and are not to be construed in any way as limiting their scope.

The PNA oligomers, nucleic acids, LNPs, and compositions thereof provided herein can be prepared from readily available starting materials using modifications to the specific synthetic protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Exemplary PNA oligomers, nucleic acids, LNPs, and compositions thereof may be prepared using any of the strategies described below.

### Example 1: Synthesis and Characterization of Exemplary PNAs

PNA oligomers were synthesized according to known methods. PNA monomers were prepared, for example, according to the methods described in Sahu et al. J. Org. Chem. 76:5614-5627 (2011). PNA oligomers were prepared using solid-phase peptide synthesis. See for example Christensen et. al. J. Pept. Sci., 1:175-183 (1995) or Bahal et. al. ChemBioChem, 13:56-60 (2012). A general procedure of Fmoc solid-phase peptide synthesis is provided below.

In an instrument or vessel capable of solid phase peptide synthesis, a resin functionalized with free amino groups (usually MBHA resin) in DMF was treated with an excess of an N-Fmoc PNA subunit dissolved in NMP, in addition to an organic base (e.g., DIPEA or MDCHA), and a coupling agent (e.g., HATU), and the mixture was incubated with the resin for 15 min. The solid-supported PNA was then washed with DCM (3×) and with DMF (3×) to remove excess reagents. The resin was then incubated with 10% piperidine (2×, 15 min each), and then once again washed with DCM (3×) and DMF (3×). Next, the resin was incubated with an additional N-Fmoc PNA subunit in a solution of NMP (0.2 M), DIEA in DMF (0.52 M), and HBTU in DMF (0.39 M) for 15 min. Excess reagents were then washed off the solid-supported PNA oligomer with DMF (4×) and DCM (lx). The above deprotection and coupling steps were repeated as many times as required to produce the PNA oligomer of the desired length and sequence. The full-length PNA oligomer was then cleaved from the solid support using TFA/m-cresol (95:5), and if necessary, could be further purified by trituration, HPLC, or column chromatography.

The sequence of exemplary PNA oligomers used in this study are:

PNA-1:
H-Lys Lys Lys j j t j t t j PEG3 C t T c T c C a C
a G g A g T c A g Lys Lys Lys-NH₂
PNA-2:
H-Lys Lys Lys t t j j t j t PEG3 t c t c c t t a a
a c c t g t c t t Lys Lys Lys-NH₂

Lys refers to the amino acid L-lysine; PEG3 is a long chain linker construct of formula: —NH—(CH₂CH₂O)₃CH₂CO—; each letter corresponds to the nucleobase in the sequence (e.g., T=thymine, j=pseudoisocytosine); a lower-case letter indicates an unsubstituted aminoethylglycine PNA subunit; an upper-case letter indicates a gamma miniPEG (—CH₂—(OCH₂CH₂)₂—OH) substituted aminooethylglycine PNA subunit (Sahu et al. vide supra).

The sequence of an exemplary load component (e.g., the donor DNA sequence) used in this study is:

SEQ ID NO: 1:
T*T*G* CCC CAC AGG GCA GTA ACG GCA GAC TTC TCC TCA
GGA GTC AGG TGC ACC ATG GTG TCT GT*T* T*G

The asterisk denotes internucleoside linkages that are phosphorothioates (instead of phosphates).

Droplet digital PCR (ddPCR) was performed with Bio-Rad QX200 using primers and probes as described below. ddPCR is a quantitative PCR method useful for the detection and measuring the amount of rare genetic variant in a DNA sample. This is achieved by partitioning DNA molecules in a sample, mixed with PCR reagents, into nanoliter-sized droplets formed in a water-oil emulsion. These individual droplets function as an individual PCR sample reaction. For the quantification of the amount of rare genetic variant in a DNA sample, the number of droplets without DNA, droplets positive for rare variant allele, and droplets positive for WT allele are measured fluorescently by the ddPCR reader, and the amount of rare variant allele is measured based on the Poisson distribution and the number of these droplets.

PNA oligomers may be characterized using many routine analytical methods. For example, PNA oligomers can be characterized using HPLC, MALDI-TOF, and/or UV-VIS.

An exemplary characterization method is as follows: a PNA stock solution was prepared using nanopore water, and the concentration was determined at 90° C. using a Cary 3 Bio spectrophotometer, with the following extinction coefficients: 13,700 M⁻¹ cm⁻¹ (A), 6,600 M⁻¹ cm⁻¹ (C), 11,700 M⁻¹ cm⁻¹ (G), and 8,600 M⁻¹ cm⁻¹ (T).

### Example 2: Preparation of Lipid Nanoparticles

General Protocol for LNPs with a 1:1 Ratio of DNA to PNA

Lipid nanoparticles (LNPs) were prepared according to the procedure outlined below. The lipids recited in Table 1 were dissolved in absolute ethanol. The PNA oligomer PNA-1 was dissolved in water (5 mg/mL), and the load component (i.e., target DNA sequence SEQ ID NO: 1) was dissolved in citrate buffer solution (0.25 mg/mL DNA concentration, using 50 mM (pH 4) citrate buffer).

TABLE 1
Exemplary LNP composition

| | Molecular | | |
|---|---|---|---|
| | Weight (unit) | Mol % | Mass % |
| Cholesterol | 386.67 | 40 | 26.16 |
| DMG-PEG2k | 2474 | 2 | 8.37 |
| DLin-MC3-DMA | 642 | 48 | 52.11 |
| DSPC | 790.17 | 10 | 13.36 |

DMG-PEG2k is 1,2-dimyristoyl-rac-glycero-3-methoxypolyethylene-2000; DLin-MC3-DMA is dilinoleylmethyl-4-dimethylaminobutyrate; DSPC is 1,2-distearoyl-sn-glycero-3-phosphocholine.

A PNA/lipid solution was prepared by adding PNA to the above lipid solution to achieve a final PNA oligomer concentration of 0.5 mg/mL and a lipid concentration of 6 mg/mL for lipids in 90% ethanol. With reference to FIG. 2, the DNA solution and PNA/lipid solution were then mixed using a syringe pump (at flow rates of 80 mL/min and 40 mL/min, respectively) through a “Tee” mixer into one stream, resulting in a decrease in ethanol concentration from 90% to 30% and the formation of a suspension [Step 1]. The resulting suspension was then mixed with a citrate buffer solution (20 mM, pH 6) through another “Tee” mixer to further lower the ethanol concentration to 10% [Step 2], and the suspension was then collected into a sterile container, and the pH was adjusted to 8 by addition of a sodium phosphate solution (500 mM, pH 8.5) [Step 3]. The suspension was loaded onto a tangential flow filtration system (with 100KD MWCO membranes) and diafiltrated against an 8× volume of DPBS at pH 7.4, and then reduced to desired concentration [Step 4]. Finally, the drug product was filtered through 0.22 μm filters and filled into sterile vials [Step 5].

General Protocol for Preparation of LNPs with 5:1 Ratio of DNA to PNA

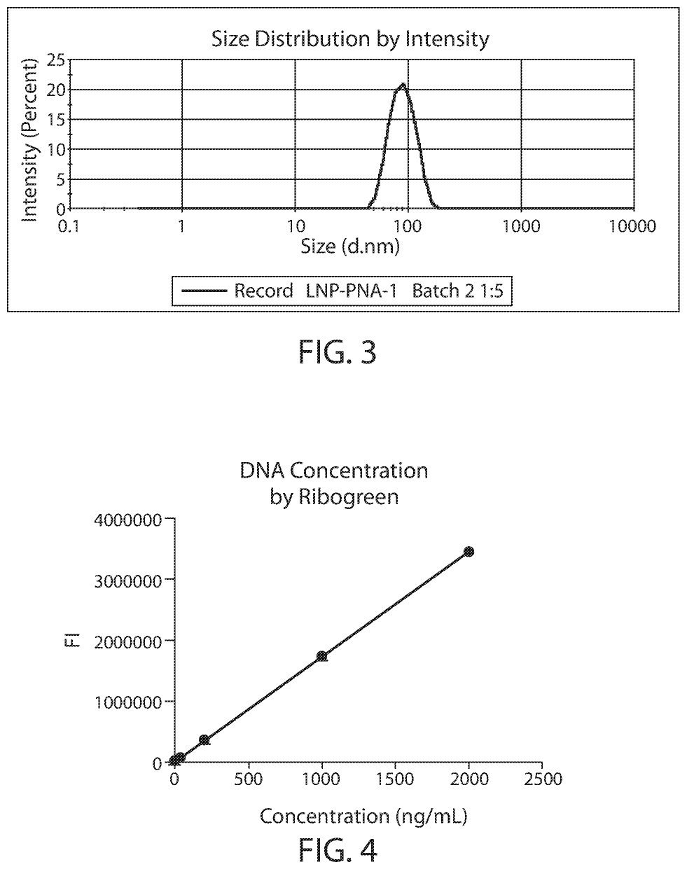

The lipid composition of Table 1 (60 mg) was dissolved in absolute ethanol (3 mL) to achieve a final concentration of 20 mg/mL. Then PNA-1 (200 μL, 5 mg/mL), water (800 μL), and absolute ethanol (6 mL) were added. Separately, SEQ ID NO: 1 (template DNA) was dissolved in a citrate buffer solution (50 mM, pH 4) to achieve a concentration of 0.25 mg/mL. With reference to FIG. 2, the DNA solution and PNA/lipid solution were then mixed through a “Tee” mixer (flow rate of 80 mL/min and 40 mL/min, respectively) [Step 1]. The resulting suspension was then diluted with citrate buffer solution (60 mL, 20 mM, pH 6) through another “Tee” mixer [Step 2]. The pH of the resulting suspension was then adjusted with sodium phosphate buffer (30 mL, 500 mM) [Step 3]. The suspension was diafiltrated against 960 mL of DPBS at pH 7.4 through a 100 kD MWCO membrane on TFF and then was further concentrated to 5 mL [Step 4]. Finally, the LNP stream was directed to a finish and fill apparatus. QC of the fluid stream indicated the average LNP particle size was about 90 nm as shown in FIG. 3.

Preparation of LNPs with Microfluidic Device

An alternate method of preparing LNPs involves use of a non-turbulent microfluidic mixing device, which provides, for example, additional capacity for fine-tuning LNP size. Exemplary LNPs were prepared using this system and the protocol described herein. LNPs were formulated with an amine-to-DNA-phosphate (N:P) ratio of 3.0. Briefly, the lipid components were dissolved in 100% ethanol at molar ratios of 40:2:48:10 (cholesterol:polyethylene glycol lipid:ionizable lipid:phosphatidylcholine). Donor DNA was dissolved in 50 mM citrate buffer (pH 4.0) while the PNA (PNA-2) was dissolved in DNAse, RNAse free water. LNPs were formed using a microfluidic mixer (NanoAssemblr Benchtop from Precision Nanosystems), in a two-step manner: (1) mixing of the DNA and PNA at a ratio of 1:1 (aqueous:aqueous) and (2) combining the DNA/PNA complex with the lipid mixtures at a ratio of 3:1 (aqueous:ethanol). After mixing, the LNPs were dialyzed against 25 mM citrate buffer (pH 4.0) for at least 4 hours and then against PBS overnight at 4° C. under gentle stirring using a 100 kDa Float-a-Lyzer G2 Dialysis Device (Repligen). The resultant formulation was then filtered through a 0.22 μm sterile filter and stored at 4° C. until use. Particle size, polydispersity and zeta-potential were measured by dynamic light scattering (DLS) using a Malvern Zetasizer DLS instrument. DNA encapsulation was determined by Oligreen assay and the encapsulation of the remaining components were quantified by HPLC.

TABLE 2a
Exemplary LNP composition

| | Molar Ratio (%) | | | |
|---|---|---|---|---|
| Sample | Sterol | PEG-lipid | Ionizable lipid | Phospholipid |
| 1 | Cholesterol (40) | DMG-PEG2k (2) | DLinDMA (48) | DSPC (10) |
| 2 | Cholesterol (40) | DMG-PEG2k (2) | C12-200 (48) | DSPC (10) |
| 3 | Cholesterol | DMG-PEG2k (2) | DLinDMA (48) | DSPC (10) |
| | Hemisuccinate (40) | | | |

TABLE 2b
Exemplary LNP Component Ratios

| | | | | |
|---|---|---|---|---|
| PNA:Donor DNA WT Ratio | 1:2 | 1:5 | 1:2 | 1:5 |
| DNA Concentration in citrate buffer | 0.5 mg/mL | 0.5 mg/mL | 0.5 mg/mL | 0.5 mg/mL |
| DNA Amount (mg) | 2.0 | 2.0 | 1.0 | 1.0 |
| PNA Concentration in water | 0.25 mg/mL | 0.1 mg/mL | 0.25 mg/mL | 0.1 mg/mL |
| PNA Amount (mg) | 1.0 | 0.4 | 0.5 | 0.2 |

### Example 3: Characterization of PNA-Loaded LNPs

### Determination of Particle Size and Polydispersity

The preparation of LNPs described in Example 2 were characterized to determine the average particle size and polydispersity using a Malvern zetasizer. Briefly, for particle size measurement, 10 μL particle suspension was added to 1 mL of water for injection (WFI) water in a transparent cuvette and the measurement was performed at 2° C. For zeta potential, a dip cell was used for the measurement.

TABLE 3
Features of exemplary LNPs

| | | | | | Cholesteryl | Cholesteryl |
|---|---|---|---|---|---|---|
| | C12-200 | C12-200 | Cholesterol | Cholesterol | Hemisuccinate | Hemisuccinate |
| | 1:5 | 1:2 | 1:5 | 1:2 | 1:5 | 1:2 |
| | PNA:DNA | PNA:DNA | PNA:DNA | PNA:DNA | PNA:DNA | PNA:DNA |
| Size (nm) | 151.6 | 150.3 | 94.21 | 92.07 | 79 | 93.56 |
| Polydispersity | 0.072 | 0.15 | 0.066 | 0.063 | 0.103 | 0.102 |
| Zeta-potential | −0.09 | 1.23 | 5.12 | 4.4 | 3.95 | 5.9 |
| at pH 4.0 (mV) | | | | | | |
| DNA Content | 16.75 | 7.87 | 27.8 | 74.7 | 44 | 31.7 μg/t |
| (μg/tube) | | | | | | |
| PNA Content | 10.34 | 10.7 | 4.85 | 19.9 | 9.01 | 11.79 |
| (μg/tube) | | | | | | |
| Ionizable | 2370 | 1550 | 180 | 490 | 310 | 210 |
| Lipid Content | | | | | | |
| (μg/tube) | | | | | | |
| Cholesterol | Below | Below | 50 | 160 | 180 | 120 |
| Content | LOQ | LOQ | | | | |
| (μg/tube) | | | | | | |

### Determination of PNA and DNA Concentrations

The average concentration of PNA oligomers and target nucleic acid (e.g., DNA) encapsulated within LNPs of an LNP preparation was determined in a number of ways. First, the concentration may be determined by deformulating the LNPs. LNPs were diluted 1000 times in 0.2% Triton X-100, then concentration of the load component (e.g., donor DNA) was directly measured using Quant-iT™ RiboGreen™ following manufacture's protocol. As the digested LNPs have no significant UV absorbance above 230 nm, the amount of DNA in the digest can be determined directly by UV absorbance. Additionally, aliquots of the digest may be analyzed by high performance liquid chromatography (HPLC) and other means (e.g., OliGreen/RiboGreen) to accurately determine the respective amount DNA in the initial particle sample. For HPLC analysis, an LNP preparation may be dissolved in 20 times volume of 2% Triton X-100, and the PNA and load component (e.g., donor DNA) are loaded on RP HPLC for quantitation.

The deformulation process was carried out as follows. LNPs were diluted 1000 times in 0.2% Triton X-100, then concentration of the load component (e.g., nucleic acid) was directly measured using Quant-iT™ RiboGreen™ following manufacture's protocol. Using a NanoDrop spectrophotometer or other small volume photometer, the spectrum and absorbance at 260 nm of the above digestion mixture (1.5 μL) was recorded against a blank sample, consisting of either MQ water or “blank” particles. Using “blank” particles ensure that materials other than PNA and DNA from the LNP digestion were not confounding or contributing to the absorbance measurements.

To estimate the total nucleic acid (i.e. PNA and DNA) an average extinction coefficient (EC) of the PNA and DNA, was used. For example, if the PNA EC was 260/μmol and the DNA EC was 600/μmol then the mean EC value (430/μmol) was used. Similarly, an average molecular weight (MW) of the two compounds was used for calculations. Using the absorbance value (A260 nm) obtained for the digested sample, the amount (μg) of PNA+DNA was calculated, and that value was divided by 10, and then divided by the amount of particles digested (mg), to obtain a crude approximation of the PNA/DNA loading in the LNPs (as a weight percent).

To more accurately determine the amount of PNA oligomer present in the LNPs, reverse-phase HPLC was performed on a C18 column, at 55° C., using a linear gradient of 0.1% TFA/water and 0.1% TFA/acetonitrile. A PNA oligomer standard curve was generated by injecting known amounts of the pure PNA oligomer on the HPLC. Typically, this was done by performing at least four injections of different amounts covering a range from 5 to 100 pmol of PNA oligomer. The area vs. pmol injected was plotted to obtain the standard curve (y=mx+b). The linear regression should have R²>0.97. To determine the amount of PNA oligomer in a sample, a sample of the above digestion mixture (approx. 50-75 pmol of total nucleic acid based on the above crude approximation) was then injected on the HPLC under the same conditions as used to generate the standard curve. For example, if the absorbance measurement for total nucleic acid provides a solution which is 1-2 nmol/mL of digest (with avg. EC of 430 OD/μmol) then about 25-50 μL of sample was injected. If the response was too low or outside the standard curve, the amount to be injected was adjusted and reinjected until a response within the standard curve was obtained. The PNA oligomer peak area of the sample from the digest was then measured and the standard curve was used to determine the pmol of PNA oligomer in the injection volume. The amount of PNA oligomer in the total volume of LNP digest could then calculated from this value.

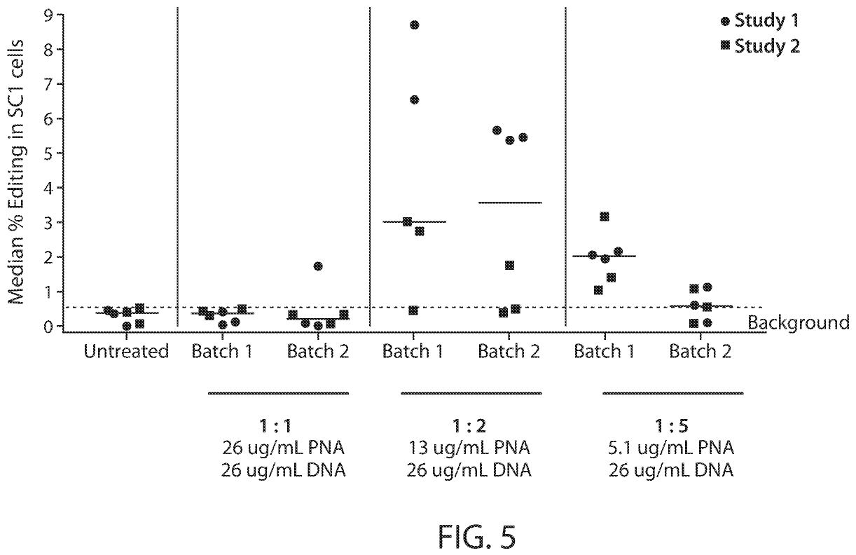

Concentration of the donor nucleic acid (i.e., DNA concentration) was determined by Oligreen or Ribogreen assay following manufacturer's protocol (see FIG. 4). The manufacturer's protocol for the OliGreen or RiboGreen assay using pure oligonucleotide was used to generate a standard curve for measuring the DNA content of the particles (see FIG. 4). Typical values of the curve will be in g. Using the total nucleic estimate (μg) from the crude approximation described above, and assuming that ˜50% of the value is DNA, a series of dilutions on a portion of the digest were performed such that one or more dilutions fell within the range of the standard curve. The diluted samples with OliGreen or RiboGreen were measured, and using the standard curve (FIG. 4), the amount of DNA was determined which could then be used to calculate the amount of DNA present in the LNPs.

### Example 4: General Methods for Use of PNA-Lipid Nanoparticles for In Vitro Gene Editing

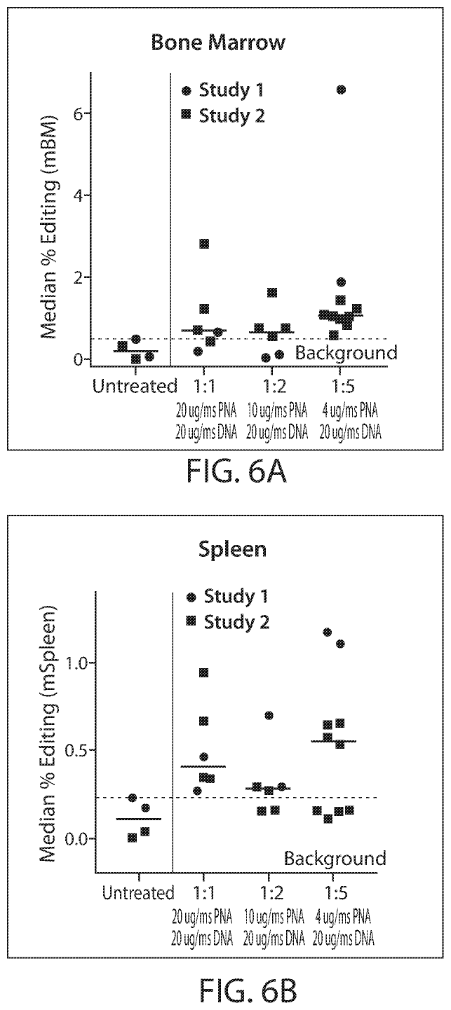

The LNPs described herein were screened for in vitro gene editing capability in a human B-cell line (SC-1) that is homozygous for the sickle cell mutation. Human SC-1 cells were cultured at density of 0.5 million cells in final volume of 0.5 mL complete media (RPMI plus 20% FBS). LNPs encapsulating a PNA oligomer (PNA-1) and donor DNA (SEQ ID NO: 1) as prepared and characterized by Examples 2 and 3 (final 0.1 mg/mL total PNA oligomer), and were added to the cells. Untreated cells were included as negative control. After 48 hours of incubation, cells were harvested, washed with phosphate-buffered saline (PBS) and subjected to whole genomic DNA extraction using Promega Wizard SV Genomic DNA purification kit. Double-stranded DNA concentration of the extract was measured fluorometrically by Qubit Fluorometer with double stranded DNA (dsDNA) High Sensitivity (HS) Assay Kit before using digital droplet PCR (ddPCR) to evaluate the percentage of gene editing (FIG. 5). Primer sequences are as follows: primer-forward (5′-CACCAACTTCATCCACGTTCAC-3′ (SEQ ID NO: 5)); primer-reverse (5′-TCTATTGCTTACATTTGCTTCTGACA-3′ (SEQ ID NO: 6). Probes are designed with 5′ Dye and 3′ minor groove binder non-fluorescent quencher (MGBNFQ): mutant (VIC®), (5′-CAGACTTCTCCACAGGA-3′); wildtype (fluorescein amidite; FAM) (5′-CAGACTTCTCCTCAGGA-3′). PCR was performed under the following conditions: 95° C., 10 min; ×40 [94° C., 30 s; 54.8° C., 4 min ramp 2° C./s]; 98° C., 10 min; 4° C. forever.

As shown in FIG. 5, LNPs prepared by the processes disclosed herein demonstrated gene correction activity in a SC-1 cell line with a gene editing activity varying by the amount of PNA:DNA encapsulated in the LNPs. Two independent studies were performed (Study 1 and Study 2 with 2 different batches of LNPs). With a 1:1 mixture of PNA:DNA, gene editing activity was of 0-2.0%. With a 1:2 mixture of PNA:DNA, gene editing activity was of 0.5-9.0%. With a 1:5 mixture of PNA:DNA, gene editing activity was of 0-3.5%.

A dose-dependent study of gene editing with LNPs in human cells may be carried out as follows: SC1 human cell line (homozygous for sickle mutation) may be cultured at density of 0.5 million cells in final volume of 0.5 mL complete media (RPMI plus 20% FBS). The cells may be treated with increasing doses of LNPs for 48 hours. Untreated cells may be included as a negative control. Cells may then be harvested, washed and then lysed to isolate whole genomic DNA. Samples may be evaluated fluorometrically (e.g., with a Qubit Fluorometer) with double stranded DNA (dsDNA) High Sensitivity (HS) Assay Kit, and could be later analyzed by ddPCR using the condition described above Gene editing may then be measured with ddPCR.

To evaluate the effect of exposure length on gene editing, the following experiment may be carried out. Cells may be harvested and prepared as described above, and could then be treated with 0.1 mg/mL of LNPs comprising PNA (PNA-1) and DNA (SEQ ID NO: 1) and harvested after 24, 48, 72, and 96 hours. After washing the genomic DNA may then be isolated from the cells and evaluated fluorometrically (e.g., with a Qubit Fluorometer). Gene editing may then be measured with ddPCR.

To measure the effect of repeated treatment on gene editing, the following experiment may be carried out. Cells may be harvested and prepared as described above, and incubated with 0.1 mg/mL LNPs comprising PNA (PNA-1) and DNA (SEQ ID NO: 1) for 48 hours. Cells may then be washed and resuspended in fresh media and treated with fresh supply of 0.1 mg/mL LNPs comprising PNA (PNA-1) and DNA (SEQ ID NO: 1) for additional 48 hours. Genomic DNA from these samples may then be prepared and evaluated by ddPCR to measure percentage of gene editing.

Gene editing may also be carried out in other cell types, such as human peripheral blood mononuclear cells (PBMCs), human CD34+ hematopoietic stem and progenitor cell (HSPC), bone marrow cells. For example, PBMCs from sickle cell anemia patients may be obtained and resuspended in complete media (e.g, m RPMI with 20% FBS) at particular density, such as 0.2×10⁶ cells/mL. The cells may then be treated with LNPs (e.g., 0.1 mg/mL), comprising PNA (PNA-1) and DNA (SEQ ID NO: 1), e.g., for 48 hours. After washing with PBS, whole genomic DNA could then be isolated, and samples may be subjected to ddPCR to determine the extent of gene editing.

For analysis of gene editing in human CD34+ hematopoietic stem and progenitor cell (HSPC) populations, PBMCs from a primary sickle cell anemia patient sample might be used to isolate CD34+ hematopoietic stem and progenitor cell (HSPC) population using positive selection magnetic beads (e.g., with Miltenyi CD34 following manufacturer's instructions). CD34+ HSPCs and remaining PBMCs depleted of CD34+ cells may then be cultured side-by-side in StemSpan SFEM II media with CD34+ Expansion Supplement (which might contain recombinant human FMS-like tyrosine kinase 3 ligand (Flt3L), stem cell factor (SCF), interleukin-3 (IL-3), interleukin-6 (IL-6), and thrombopoietin (TPO)). Later, LNPs comprising PNA (PNA-1) and DNA (SEQ ID NO: 1) may be added to cells at final 0.1 mg/mL API and be incubated for 48 hours. Untreated samples may be included as negative controls. Cells may then be collected and washed for genomic DNA extraction, and gene editing may be measured using ddPCR.

Gene editing in mouse bone marrow cells may be measured. For example, bone marrow cells from Townes sickle cell mice may be obtained and resuspended in complete media (RPMI with 20% FBS) at density of 0.2×10⁶ cells/mL, and may then be treated with 0.1 mg/mL LNPs comprising PNA (PNA-1) and DNA (SEQ ID NO: 1) for 48 hours. After washing with PBS, whole genomic DNA could then be isolated, and samples may be subjected to ddPCR to determine the extent of gene editing.

The correction of sickle cell mutation may be confirmed by next generation sequencing (NGS). Amplicons could be prepared using PCR and primers designed around SCD mutation in human hemoglobin gene (Forward: 5′-TTGTAACCTTGATACCAACC-3′ (SEQ ID NO: 8) and Reverse: 5′-CTTACATTTGCTTCTGACAC-3′ (SEQ ID NO: 9), PCR conditions: 95° C., 3 min; ×35 [95° C., 30 s; 49.6° C., 30 s; 72° C., 1 min]; 72° C., 10 min; 4° C. forever). PCR products could be subjected to column clean-up (QIAquick Qiagen) and amplicon may be evaluated on Qubit and later on 2% gel for size and purity. NGS analysis of the samples may be performed by a fee for service provider on a blind basis using the Illumina TruSeq Paired-End Sequencing workflow. Samples may then be sequenced on Illumina MiSeq (2×150 bp) platform (merged paired reads). Unique nucleotide sequences in the region of interest could be identified and a relative abundance may be calculated for each unique sequence. In edited samples, variant abundance of unique sequences with correction of mutant GTG to wild-type GAG may then be calculated.

### Example 5: General Methods for Use of PNA-Lipid Nanoparticles for In Vivo Gene Editing

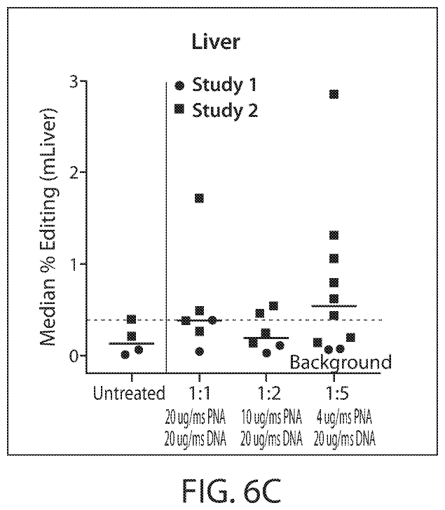

In order to evaluate the extent of gene editing of the LNPs described herein, further experiments were carried out in a mouse model of sickle cell anemia. Sickle cell Townes mice were treated with a single dose of LNPs containing a ratio of either 1:1, 1:2, or 1:5 PNA oligomer (PNA-1) to a template DNA sequence (SEQ ID NO: 1). Two independent studies were performed, wherein the population of mice tested in the second study was larger than in the first. NPs were administered into mice via intravenous tail vein intravenous injection. Ten days after administration, the mice were euthanized, and their bone marrow, spleen, and liver cells were harvested. Cells from these tissues were then lysed to isolate whole genomic DNA and DNA samples were evaluated fluorometrically by a Qubit Fluorometer with double stranded DNA (dsDNA) High Sensitivity (HS) Assay Kit as described above. The level of gene editing in these DNA samples were analyzed by ddPCR using the condition described above. FIGS. 6A-6C illustrate the degree of gene editing in vivo, with a median editing level ranging from 0-6% in bone marrow cells (FIG. 6A), 0-1.2% in spleen cells (FIG. 6B), and 0-3% in liver cells (FIG. 6C). Based on these studies, the LNP containing 1:5 ratio of PNA-DNA provided the highest degree of gene editing.

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A lipid nanoparticle (LNP) comprising:
a) one or more or all of:
(i) an ionizable lipid;
(ii) a phospholipid;
(iii) a sterol; and
(iv) an alkylene glycol-containing lipid; and
b) a neutral or positively charged nucleic acid mimic (NPNAM).

2. The LNP of claim 1, wherein the NPNAM comprises a PNA oligomer.

3. The LNP of claim 2, wherein the PNA oligomer comprises a tail-clamp PNA oligomer (tcPNA).

4. The LNP of any of the preceding claims, wherein the PNA oligomer comprises a gamma-substituted PNA subunit.

5. The LNP of claim 4, wherein the gamma-substituted PNA subunit comprises a polyethylene glycol moiety at the gamma position.

6. The LNP of any of the preceding claims, wherein the PNA oligomer comprises a PNA subunit having a structure of Formula (I): 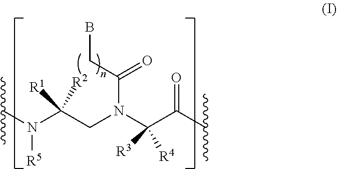
wherein:
B is a nucleobase;
each of R¹, R², R³, and R⁴ is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, and heteroaryl is optionally substituted with one or more R⁶
R⁵ is hydrogen or alkyl;
each R⁶ is independently alkyl, heteroalkyl, amino, halo, oxo, or hydroxy;
n is an integer between 1 and 10; and
each “ [symbol] ” is independently N-terminus of the PNA oligomer, the C-terminus of the PNA oligomer, or an attachment point to another PNA subunit.

7. The LNP of claim 6, wherein one of R¹ and R² comprises a C2-C30 heteroalkyl.

8. The LNP of any one of claims 6-7, wherein one of R¹ and R² comprises a C2-C30 heteroalkyl and the other of R¹ and R² is hydrogen.

9. The LNP of any one of claims 6-8, wherein the C2-C30 heteroalkyl comprises a C2-C30 polyalkylene glycol (e.g., a C2-C30 polyethylene glycol).

10. The LNP of any one of claims 6-9, wherein R¹ comprises a C2-C30 polyethylene glycol (e.g., R1 is a structure of Formula (VI-a) or (VI-b) as described herein).

11. The LNP of any one of claims 6-10, wherein each of R³, R⁴, and R⁵ is independently hydrogen.

12. The LNP of any one of claims 6-11, wherein B is selected from adenine, cytosine, guanine, thymine, uracil, pseudoisocytosine, 2,6-diaminopurine, 2-thiouracil, 7-deazaadenine, and 7-deazaguanine; R¹ is —CH₂O—[CH₂CH₂O]ₑ—R₇ wherein e is 0, 1, 2, 3 or 4; R₇ is H, methyl, ethyl or t-butyl; each of R², R³, R⁴, and R⁵ is independently hydrogen; and n is 1.

13. The LNP of any one of claims 6-11, wherein B is selected from adenine, cytosine, guanine, thymine, uracil, pseudoisocytosine, 2,6-diaminopurine, 2-thiouracil, 7-deazaadenine, and 7-deazaguanine; R² is —CH₂O—[CH₂CH₂O]ₑ—R₇ wherein e is 0, 1, 2, 3 or 4; R₇ is hydrogen, methyl, ethyl or t-butyl; each of R¹, R³, R⁴, and R⁵ is independently hydrogen; and n is 1.

14. The LNP of any one of claims 6-11, wherein B is selected from adenine, cytosine, guanine, thymine, uracil, pseudoisocytosine, 2,6-diaminopurine, 2-thiouracil, 7-deazaadenine, and 7-deazaguanine; R³ is a —CH₂O—[CH₂CH₂O]ₑ—R₇ wherein e is 0, 1, 2, 3 or 4; R₇ is hydrogen, methyl, ethyl or t-butyl; each of R¹, R², R⁴, and R⁵ is independently hydrogen; and n is 1.

15. The LNP of any one of claims 6-11, wherein B is selected from adenine, cytosine, guanine, thymine, uracil, pseudoisocytosine, 2,6-diaminopurine, 2-thiouracil, 7-deazaadenine, and 7-deazaguanine; R⁴ is —CH₂O—[CH₂CH₂O]ₑ—R₇ wherein e is 0, 1, 2, 3 or 4; R₇ is hydrogen, methyl, ethyl or t-butyl; each of R¹, R², R³, and R⁵ is independently hydrogen; and n is 1.

16. The LNP of any one of claims 6-15, wherein B comprises a naturally occurring nucleobase (e.g., adenine, cytosine, guanine, thymine, uracil).

17. The LNP of any one of claims 6-15, wherein B comprises a non-naturally occurring nucleobase (e.g., pseudoisocytosine, 2,6-diaminopurine, 2-thiouracil, 7-deaza adenine, and 7-deazaguanine).

18. The LNP of any one of claims 6-11, wherein n is 1 or 2 (e.g., n is 1).

19. The LNP of any one of claims 6-11, wherein B is selected from adenine, cytosine, guanine, thymine, uracil, pseudoisocytosine, 2,6-diaminopurine, 2-thiouracil, 7-deaza adenine and 7-deazaguanine; R¹ comprises a polyethylene glycol (e.g., a C2-C30 polyethylene glycol); each of R², R³, R⁴, and R⁵ is independently hydrogen; and n is 1.

20. The LNP of any one of the preceding claims, the PNA oligomer comprises greater than 2, 3, 4, 5, 6, 7, 8, 9, 10, 15, 20, 25, 30, 35, 40, 45, or 50 PNA monomer subunits.

21. The LNP of any one of the preceding claims, wherein the PNA oligomer comprises a PNA subunit having a structure of Formula (I-a): 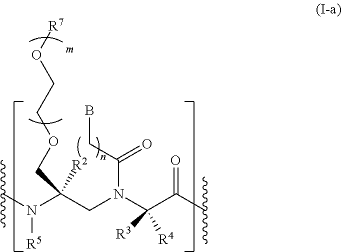
wherein:
B is a nucleobase;
each of R², R³, and R⁴ is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, and heteroaryl is optionally substituted with one or more R⁶;
R⁵ is hydrogen or alkyl;
each R⁶ is independently alkyl, heteroalkyl, amino, halo, oxo, or hydroxy;
R⁷ is hydrogen or alkyl;
each of m and n is an integer between 1 and 10; and
each “ [symbol] ” is independently N-terminus of the PNA oligomer, the C-terminus of the PNA oligomer, or an attachment point to another PNA subunit.

22. The LNP of claim 21, wherein B is selected from adenine, cytosine, guanine, thymine, uracil, pseudoisocytosine, 2,6-diaminopurine, 2-thiouracil, 7-deazaadenine, and 7-deazaguanine; each of R², R³, R⁴, R⁵, and R⁷ is independently hydrogen; m is 2 and n is 1.

23. The LNP of any one of claims 1-20, wherein the PNA oligomer comprises a PNA subunit having a structure of Formula (I-b): 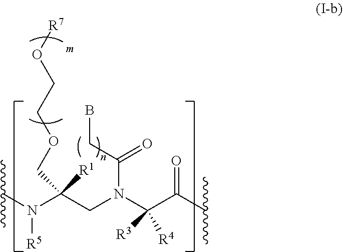
wherein:
B is a nucleobase;
each of R¹, R³, and R⁴ is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, and heteroaryl is optionally substituted with one or more R⁶;
R⁵ is hydrogen or alkyl;
each R⁶ is independently alkyl, heteroalkyl, amino, halo, oxo, or hydroxy;
R⁷ is hydrogen or alkyl;
each of m and n is an integer between 1 and 10; and
each “ [symbol] ” is independently N-terminus of the PNA oligomer, the C-terminus of the PNA oligomer, or an attachment point to another PNA subunit.

24. The LNP of claim 23, wherein B is selected from adenine, cytosine, guanine, thymine, uracil, pseudoisocytosine, 2,6-diaminopurine, 2-thiouracil, 7-deazaadenine, and 7-deazaguanine; each of R¹, R³, R⁴, R⁵, and R⁷ is independently hydrogen; m is 2 and n is 1.

25. The LNP of any one of the preceding claims, wherein the PNA oligomer comprising a structure of Formula (I), Formula (I-a), or Formula (I-b) is a tail-clamp PNA (tcPNA).

26. The LNP of any one of the preceding claims, wherein the PNA oligomer comprises a PNA sequence selected from PNA-1 or PNA-2.

27. The LNP of any one of the preceding claims, wherein the amount of PNA oligomer encapsulated and/or entrapped within the LNP is between 0.1% to 50% (e.g., 1% to 25%, 1% to 10%, or 2% to 5%) by weight of PNA oligomers to the total weight of the LNP.

28. The LNP of any one of the preceding claims, wherein the LNP further comprises a load component, e.g., encapsulated and/or entrapped within the LNP.

29. The LNP of claim 28, wherein the load component comprises a nucleic acid (e.g., a DNA, e.g., single-stranded DNA).

30. The LNP of claim 29, wherein the nucleic acid comprises DNA.

31. The LNP of any one of claims 29-30, wherein the nucleic acid comprises between 20 and 100 nucleotides.

32. The LNP of any one of claims 29-31, wherein the nucleic acid comprises a phosphorothioate linkage.

33. The LNP of any one of claims 29-32, wherein the nucleic acid comprises a phosphorothioate linkage at the 3′ terminus or 5′ terminus.

34. The LNP of claim 32, wherein the nucleic acid comprises at least one phosphorothioate linkage at both the 3′ terminus and the 5′ terminus.

35. The LNP of any one of claims 32-34, wherein the nucleic acid comprises a phosphorothioate linkage between the 5′-terminal nucleotide (5-1) and the immediately adjacent nucleotide (5-2).

36. The LNP of any one of claims 32-35, wherein the nucleic acid comprises a phosphorothioate linkage between the 5-2 nucleotide and the immediately adjacent downstream nucleotide (5-3).

37. The LNP of any one of claims 32-36, wherein the nucleic acid comprises a phosphorothioate linkage the between the 5-3 nucleotide and the immediately adjacent downstream nucleotide (5-4).

38. The LNP of any one of claims 32-37, wherein the nucleic acid comprises a phosphorothioate linkage between the 3′-terminal nucleotide (3-1) and the immediately adjacent nucleotide (3-2).

39. The LNP of any one of claims 32-38, wherein the nucleic acid comprises a phosphorothioate linkage between the 3-2 nucleotide and the immediately adjacent upstream nucleotide (3-3).

40. The LNP of any one of claims 32-39, wherein the nucleic acid comprises a phosphorothioate linkage the between the 3-3 nucleotide and the immediately adjacent upstream nucleotide (3-4).

41. The LNP of any one of claims 32-40, wherein the nucleic acid comprises at least two phosphorothioate linkages at each of its 3′ and 5′ termini.

42. The LNP of any one of claims 32-41, wherein the nucleic acid comprises an antisense agent, an mRNA, or an siRNA.

43. The LNP of any one of claims 32-42, wherein the load component comprises a nucleic acid having a sequence which is the same or the complement of a sequence to which the PNA oligomer has Watson Crick homology.

44. The LNP of any of claims 32-43, wherein the load component comprises a nucleic acid having a sequence which is the same or the complement of a sequence to which the PNA oligomer has Hoogsteen homology.

45. The LNP of any of claims 32-44, wherein the load component comprises a nucleic acid having a sequence of at least 2, 5, 10, or 20 bases which is the same or the complement of a sequence that is within 1,000, 500, or 200 base pairs of a sequence to which the PNA oligomer has Watson Crick homology.

46. The LNP of any of claims 32-45, wherein the load component comprises a nucleic acid having a sequence of at least 2, 5, 10, or 20 bases which is the same or the complement of a sequence that is within 1,000, 500, or 200 base pairs of a sequence to which the PNA oligomer has Hoogsteen homology.

47. The LNP of any one of the preceding claims, wherein the lipid comprises an ionizable lipid.

48. The LNP of claim 47, wherein the ionizable lipid comprises a cationic lipid or an anionic lipid.

49. The LNP of any one of claims 47-48, wherein the ionizable lipid comprises a structure of Formula (II): 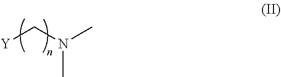
or a pharmaceutically acceptable salt thereof, wherein:
Y is 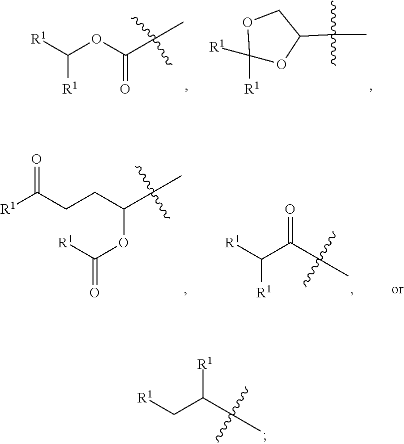
each R¹ is independently alkyl, alkenyl, alkynyl, or heteroalkyl, each of which is optionally substituted with R^{A};
each R^{A} is independently alkyl, halo, hydroxy, amino, cycloalkyl, or heterocyclyl; and
n is an integer between 1 and 6.

50. The LNP of claim 49, wherein Y is 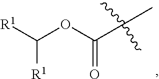
each R¹ is independently a C18 alkenyl (e.g., linoleyl), and n is 3.

51. The LNP of any one of claims 47-50, wherein the ionizable lipid is selected from DLin-MC3-DMA, DLin-KC2-DMA, DLin-DMA, DLin-K-DMA, DLin-DAP, 98N12-5, C12-200, and DODMA, or a pharmaceutically acceptable salt thereof.

52. The LNP of any one of claims 47-51, wherein the ionizable lipid comprises dilinoleylmethyl-4-dimethylaminobutyrate (DLin-MC3-DMA).

53. The LNP of any one of claims 47-51, wherein the ionizable lipid comprises DLin-DMA.

54. The LNP of any one of claims 47-51, wherein the ionizable lipid comprises C12-200.

55. The LNP of any one of claims 47-54, wherein the ionizable lipid is present in the LNP at a concentration greater than about 0.1 mol % (e.g., greater than about 0.5 mol %, about 1 mol %, about 5 mol %, about 10 mol %, about 15 mol %, about 20 mol %, about 25 mol %, about 35 mol %, about 40 mol %, about 45 mol %, about 50 mol %, about 55 mol %, about 60 mol %, about 65 mol %, or about 70%) of the total lipid concentration of the LNP.

56. The LNP of any one of claims 47-55, wherein the ionizable lipid is present in the LNP at a concentration between about 1 mol % to about 95 mol % (e.g. between about 5 mol % to about 90 mol %, about 10 mol % to about 70 mol %, about 20 mol % to about 80 mol %, about 30 mol % to about 70 mol %, about 40 mol % to about 60 mol %, about 40 mol % to 50 mol %, or about 50 mol % to 60 mol %) of the total lipid concentration of the LNP.

57. The LNP of any one of the preceding claims, further comprising an additional lipid.

58. The LNP of claim 57, wherein the additional lipid comprises a phospholipid, a sterol, or an alkylene glycol-containing lipid (e.g., a PEG-containing lipid).

59. The LNP of claim 58, wherein the additional lipid comprises a phospholipid.

60. The LNP of claim 59, wherein the phospholipid is a naturally occurring or synthetic phospholipid.

61. The LNP of any one of 59-60, wherein the phospholipid comprises a structure of Formula (III): 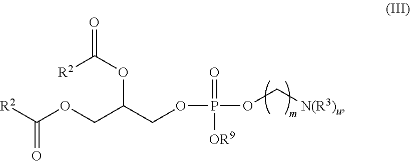
or a pharmaceutically acceptable salt thereof, wherein:
each R² is independently alkyl, alkenyl, or heteroalkyl;
each R³ is independently hydrogen or alkyl;
R⁹ is absent, hydrogen, or alkyl;
each R^{B} is independently alkyl, halo, hydroxy, amino, cycloalkyl, or heterocyclyl;
m is an integer between 1 and 4; and
u is an integer between 2 and 3.

62. The LNP of claim 61, wherein R³ is methyl, each R² is independently alkyl (e.g., heptadecyl), and m is 2.

63. The LNP of any one of claims 59-62, wherein the phospholipid comprises a phosphocholine.

64. The LNP of any one of claims 59-63, wherein the phospholipid comprises DMPC, DSPC, DOPC, DPPC, and DOPE.

65. The LNP of any one of claims 59-64, wherein the phospholipid is 1,2-distearoyl-sn-glycero-3-phosphocholine (DSPC).

66. The LNP of any one of claims 59-65, wherein the phospholipid is present in the LNP at a concentration greater than about 0.1 mol % (e.g., greater than about 0.5 mol %, about 1 mol %, about 2 mol %, about 3 mol %, about 4 mol %, about 5 mol %, about 6 mol %, about 7 mol %, about 8 mol %, about 9 mol %, about 10 mol %, about 12.5 mol %, about 15 mol %, or about 20 mol %) of the total lipid concentration of the LNP.

67. The LNP of any one of claims 59-66, wherein the phospholipid is present in the LNP at a concentration between about 0.1 mol % to about 50 mol % (e.g., between about 0.5 mol % to about 40 mol %, about 1 mol % to about 30 mol %, about 2.5 mol % to about 20 mol %, about 5 mol % to about 10 mol %) of the total lipid concentration of the LNP.

68. The LNP of claim 57, wherein the additional lipid comprises a sterol.

69. The LNP of claim 68, wherein the sterol is a naturally occurring sterol.

70. The LNP of any one of claims 68-69, wherein the sterol comprises a structure of Formula (IV): 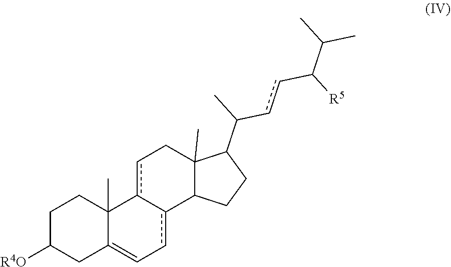
or a pharmaceutically acceptable salt thereof, wherein:
R⁴ is hydrogen, alkyl, heteroalkyl, or —C(O)R^{C};
R⁵ is hydrogen, alkyl, or —OR^{D};
each of R^{C} and R^{D} is independently hydrogen, alkyl, alkenyl, heteroalkyl, cycloalkyl, heterocyclyl, aryl or heteroaryl, wherein each alkyl, alkenyl, heteroalkyl, cycloalkyl, heterocyclyl, aryl or heteroaryl is optionally substituted with alkyl, halo, or oxo; and
each “ [symbol] ” is either a single or double bond, wherein each carbon atom participating in the single or double bond is bound to 0, 1, or 2 hydrogens, valency permitting.

71. The LNP of claim 70, wherein R⁴ is hydrogen, R⁵ is hydrogen or alkyl (e.g., hydrogen), and each [symbol] denotes a single bond.

72. The LNP of any one of claims 68-71, wherein the sterol comprises cholesterol, cholesterol hemisuccinate, dehydroergosterol, ergosterol, campesterol, sitosterol, and stigmasterol.

73. The LNP of any one of claims 68-72, wherein the sterol is cholesterol.

74. The LNP of any one of claims 68-72, wherein the sterol is cholesterol hemisuccinate.

75. The LNP of any one of claims 68-74, wherein the sterol is present in the LNP at a concentration greater than about 0.1 mol % (e.g., greater than about 0.5 mol %, about 1 mol %, about 5 mol %, about 10 mol %, about 15 mol %, about 20 mol %, about 25 mol %, about 35 mol %, about 40 mol %, about 45 mol %, about 50 mol %, about 55 mol %, about 60 mol %, about 65 mol %, or about 70%) of the total lipid concentration of the LNP.

76. The LNP of any one of claims 68-75, wherein the sterol is present in the LNP at a concentration between about 1 mol % to about 90 mol % (e.g. between about 5 mol % to about 80 mol %, about 10 mol % to about 70 mol %, about 20 mol % to about 60 mol %, about 30 mol % to about 50%, about 40% to about 50 mol %, or about 30 mol % to 40 mol %) of the total lipid concentration of the LNP.

77. The LNP of claim 57, wherein the additional lipid comprises an alkylene-containing lipid (e.g., a PEG-containing lipid).

78. The LNP of claim 77, wherein the alkylene glycol-containing lipid is a PEG-containing lipid.

79. The LNP of claim 78, wherein the PEG-containing lipid comprises a PEG moiety between 200 and 10,000 Da.

80. The LNP of any one of claims 78-79, wherein the PEG-containing lipid comprises a structure of Formula (V): 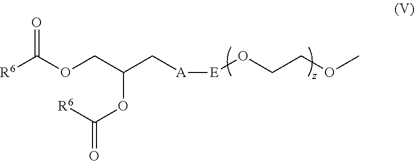
or a pharmaceutically acceptable salt thereof, wherein:
each R⁶ is independently alkyl, alkenyl, or heteroalkyl, each of which is optionally substituted with R^{E};
A is absent, O, CH₂, C(O), or NH;
E is absent, alkyl, or heteroalkyl, wherein alkyl or heteroalkyl is optionally substituted with oxo;
each R^{E} is independently alkyl, halo, hydroxy, amino, cycloalkyl, or heterocyclyl; and
z is an integer between 10 and 200.

81. The LNP of claim 80, wherein each R⁶ is independently alkyl (e.g., tridecyl), A and E are absent, and z is 45.

82. The LNP of any one of claims 78-81, wherein the PEG-containing lipid comprises PEG-c-DOMG, PEG-DSG, PEG-DPG, or PEG-DMG.

83. The LNP of any one of claims 78-82, wherein the PEG-containing lipid comprises PEG-DMG (e.g., DMG-PEG2k).

84. The LNP of any one of claims 78-83, wherein the PEG-containing lipid is present in the LNP at a concentration greater than about 0.01 mol % (e.g., greater than about 0.05 mol %, about 0.1 mol %, about 0.5 mol %, about 1 mol %, about 1.5 mol %, about 2 mol %, about 2.5 mol %, about 3 mol %, about 3.5 mol %, about 4 mol %, about 4.5 mol %, about 5 mol %, about 5.5 mol %, about 6 mol %, about 6.5 mol %, about 7 mol %, about 7.5 mol %, about 8 mol %, about 9 mol %, about 10 mol %, about 12.5 mol %, about 15 mol %, or about 20 mol %) of the total lipid concentration of the LNP.

85. The LNP of any one of claims 78-84, wherein the PEG-containing lipid is present in the LNP at a concentration between about 0.1 mol % to about 50 mol % (e.g. between about 0.5 mol % to about 40 mol %, about 1 mol % to about 30 mol %, about 2.5 mol % to about 20 mol %, about 5 mol % to about 10 mol %) of the total lipid concentration of the LNP.

86. The LNP of any one of the preceding claims, wherein the LNP comprises at least two of an ionizable lipid, a phospholipid, a sterol, and a PEG-containing lipid.

87. The LNP of any one of the preceding claims, wherein the LNP comprises at least three of an ionizable lipid, a phospholipid, a sterol, and a PEG-containing lipid.

88. The LNP of any one of the preceding claims, wherein the LNP comprises each of an ionizable lipid, a phospholipid, a sterol, and a PEG-containing lipid.

89. The LNP of any one of the preceding claims, wherein the LNP comprises each of:
(i) an ionizable lipid at a concentration between about 1 mol % to about 95 mol % (e.g. about 20 mol % to about 80 mol %);
(ii) a phospholipid at a concentration between 0.1 mol % to about 50 mol % (e.g. between about 2.5 mol % to about 20 mol %);
(iii) a sterol at a concentration between about 1 mol % to about 95 mol % (e.g. about 20 mol % to about 80 mol %); and
(iv) a PEG-containing lipid at a concentration between about 0.1 mol % to about 50 mol % (e.g. between about 2.5 mol % to about 20 mol %).

90. The LNP of any one of the preceding claims, wherein the LNP comprises at least two of DLin-MC3-DMA, DSPC, cholesterol, and DMG-PEG2k.

91. The LNP of any one of the preceding claims, wherein the LNP comprises at least three of DLin-MC3-DMA, DSPC, cholesterol, and DMG-PEG2k.

92. The LNP of any one of the preceding claims, wherein the LNP comprises each of DLin-MC3-DMA, DSPC, cholesterol, and DMG-PEG2k.

93. The LNP of any one of the preceding claims, wherein the LNP comprises at least two of DLin-DMA, DSPC, cholesterol, and DMG-PEG2k.

94. The LNP of any one of the preceding claims, wherein the LNP comprises at least three of DLin-DMA, DSPC, cholesterol, and DMG-PEG2k.

95. The LNP of any one of the preceding claims, wherein the LNP comprises each of DLin-DMA, DSPC, cholesterol, and DMG-PEG2k.

96. The LNP of any one of the preceding claims, wherein the LNP comprises at least two of C12-200, DSPC, cholesterol, and DMG-PEG2k.

97. The LNP of any one of the preceding claims, wherein the LNP comprises at least three of C12-200, DSPC, cholesterol, and DMG-PEG2k.

98. The LNP of any one of the preceding claims, wherein the LNP comprises each of C12-200, DSPC, cholesterol, and DMG-PEG2k.

99. The LNP of any one of the preceding claims, wherein the LNP comprises at least two of DLin-DMA, DSPC, cholesterol hemisuccinate, and DMG-PEG2k.

100. The LNP of any one of the preceding claims, wherein the LNP comprises at least three of DLin-DMA, DSPC, cholesterol hemisuccinate, and DMG-PEG2k.

101. The LNP of any one of the preceding claims, wherein the LNP comprises each of DLin-DMA, DSPC, cholesterol hemisuccinate, and DMG-PEG2k.

102. The LNP of any one of the preceding claims, wherein the LNP comprises each of:
(i) DLin-MC3-DMA at a concentration between about 1 mol % to about 95 mol % (e.g. about 20 mol % to about 80 mol %);
(ii) DSPC at a concentration between 0.1 mol % to about 50 mol % (e.g. between about 2.5% to about 20 mol %);
(iii) cholesterol at a concentration between about 1 mol % to about 95 mol % (e.g. about 20 mol % to about 80 mol %); and
(iv) DMG-PEG2k at a concentration between about 0.1 mol % to about 50 mol % (e.g. between about 2.5 mol % to about 20 mol %).

103. The LNP of any one of the preceding claims, wherein the LNP comprises each of:
(i) DLin-DMA at a concentration between about 1 mol % to about 95 mol % (e.g. about 20 mol % to about 80 mol %);
(ii) DSPC at a concentration between 0.1 mol % to about 50 mol % (e.g. between about 2.5% to about 20 mol %);
(iii) cholesterol at a concentration between about 1 mol % to about 95 mol % (e.g. about 20 mol % to about 80 mol %); and
(iv) DMG-PEG2k at a concentration between about 0.1 mol % to about 50 mol % (e.g. between about 2.5 mol % to about 20 mol %).

104. The LNP of any one of the preceding claims, wherein the LNP comprises each of:
(i) DLin-DMA at a concentration between about 1 mol % to about 95 mol % (e.g. about 20 mol % to about 80 mol %);
(ii) DSPC at a concentration between 0.1 mol % to about 50 mol % (e.g. between about 2.5% to about 20 mol %);
(iii) cholesterol hemisuccinate at a concentration between about 1 mol % to about 95 mol % (e.g. about 20 mol % to about 80 mol %); and
(iv) DMG-PEG2k at a concentration between about 0.1 mol % to about 50 mol % (e.g. between about 2.5 mol % to about 20 mol %).

105. The LNP of any one of the preceding claims, wherein the LNP comprises each of:
(i) C12-200 at a concentration between about 1 mol % to about 95 mol % (e.g. about 20 mol % to about 80 mol %);
(ii) DSPC at a concentration between 0.1 mol % to about 50 mol % (e.g. between about 2.5% to about 20 mol %);
(iii) cholesterol at a concentration between about 1 mol % to about 95 mol % (e.g. about 20 mol % to about 80 mol %); and
(iv) DMG-PEG2k at a concentration between about 0.1 mol % to about 50 mol % (e.g. between about 2.5 mol % to about 20 mol %).

106. The LNP of any one of the preceding claims, wherein the LNP comprises one or more of the following properties:
(i) the amount of PNA oligomer encapsulated and/or entrapped within the LNP is greater than or equal to 2 percent (2%) by weight of PNA oligomer to the total weight of the LNP;
(ii) the diameter of the LNP is between 30 to 200 nanometers; or
(iii) the LNP further comprises a load component (e.g., a nucleic acid), e.g., wherein the amount of the load component encapsulated and/or entrapped within the LNP is greater than or equal to 0.5 percent (0.5%) by weight of load component to the total weight of the LNP.

107. The LNP of claim 106, comprising property (i).

108. The LNP of claim 106, comprising property (ii).

109. The LNP of claim 106, comprising properties (i) and (ii).

110. A lipid nanoparticle (LNP) comprising:
(i) an ionizable lipid;
(ii) a phospholipid;
(iii) a sterol (e.g., cholesterol);
(iv) a PEG-containing lipid; and
a neutral or positively charged nucleic acid mimic (NPNAM).

111. The LNP of claim 110, wherein the NPNAM comprises a PNA oligomer, a morpholino, a pyrrolidine-amide oligonucleotide mimic, a morpholinoglycine oligonucleotide, or a methyl phosphonate.

112. The LNP of claim 111, wherein the NPNAM comprises a PNA oligomer.

113. The LNP of claim 112, wherein the PNA oligomer is a tail-clamp PNA oligomer (tcPNA).

114. The LNP of any one of the preceding claims, made by a method described herein.

115. A preparation comprising a plurality of LNPs, wherein each LNP of the plurality comprises:
a) one or more or all of:
(i) an ionizable lipid;
(ii) a phospholipid;
(iii) a sterol (e.g., cholesterol); and
(iv) an alkylene glycol-containing lipid; and
b) a neutral or positively charged nucleic acid mimic (NPNAM).

116. The preparation of claim 115, wherein the NPNAM comprises a PNA oligomer.

117. The preparation of claim 116, wherein the PNA oligomer comprises a tail-clamp PNA oligomer (tcPNA).

118. The preparation of any one of claims 116-117, wherein the PNA oligomer comprises a gamma-substituted PNA subunit.

119. The preparation of claim 118, wherein the gamma-substituted PNA subunit comprises a polyethylene glycol moiety at the gamma position.

120. The preparation of any one of claims 115-119, wherein the PNA oligomer comprises a PNA subunit having a structure of Formula (I): 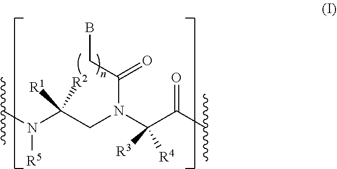
wherein:
B is a nucleobase;
each of R¹, R², R³, and R⁴ is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, and heteroaryl is optionally substituted with one or more R⁶
R⁵ is hydrogen or alkyl;
each R⁶ is independently alkyl, heteroalkyl, amino, halo, oxo, or hydroxy;
n is an integer between 1 and 10; and
each “ [symbol] ” is independently N-terminus of the PNA oligomer, the C-terminus of the PNA oligomer, or an attachment point to another PNA subunit.

121. The preparation of any one of claims 115-120, comprising an LNP of any one of claims 1-114.

122. The preparation of any one of claims 115-121, wherein the preparation comprises one of the following properties:
(i) the amount of PNA oligomer encapsulated and/or entrapped within each LNP of the preparation is greater than or equal to 0.05% by weight of PNA oligomer to the total weight of the LNP;
(ii) at least 5% of the LNPs of the preparation have an average diameter of between 5 and 500 nm;
(iii) the preparation contains less than 0.05% by weight of free LNPs, free lipid, or a PNA oligomer; and
(iv) the preparation contains less than 0.05% by weight of empty LNPs.

123. The preparation of claim 122, comprising two of properties (i)-(iv).

124. The preparation of claim 122, comprising three of properties (i)-(iv).

125. The preparation of claim 122, comprising all of properties (i)-(iv).

126. The preparation of claim 122, comprising property (i).

127. The preparation of claim 122, comprising property (ii).

128. The preparation of claim 122, comprising property (iii).

129. The preparation of claim 122, comprising property (iv).

130. The preparation of any of claims 115-129, wherein the preparation is a pharmaceutically acceptable preparation.

131. The preparation of any of claims 115-130, disposed in a delivery device (e.g., a cannula, cannula, a syringe, a depot, a pump, or a tube).

132. The preparation of any of claims 115-131, disposed in a storage device (e.g., a vial).

133. A method comprising:
a) combining a first solution and a second solution at a junction under conditions suitable to produce formation of a lipid nanoparticle (LNP) in a post-junction fluid stream comprising post-junction fluid, wherein:
(i) the first solution comprises water or an aqueous solution or buffer; and
(ii) the second solution comprises: (a′) a neutral or positively charged nucleic acid mimic (NPNAM); (b′) a lipid; and (c′) a water miscible organic solvent;
b) forming an LNP comprising the NPNAM and the lipid in the post-junction fluid stream.

134. The method of claim 133, wherein the NPNAM is encapsulated and/or entrapped in the LNP.

135. The method of any one of claims 133-134, wherein the post junction fluid stream comprises the first solution and the second solution and the NPNAM is encapsulated and/or entrapped in the LNP.

136. The method of any one of claims 133-135, wherein the post junction fluid stream is made by combining the first solution and the second solution.

137. The method of any one of claims 133-136, wherein the NPNAM is selected from a peptide nucleic acid, morpholino, pyrrolidine-amide oligonucleotide mimic, morpholinoglycine oligonucleotide and methyl phosphonate.

138. The method of any one of claims 133-137, wherein the NPNAM is a peptide nucleic acid (PNA) oligomer.

139. The method of claim 138, wherein the LNP is an LNP of any one of claims 1-114.

140. The method of any one of claims 133-139, wherein said second solution comprises a second NPNAM.

141. The method of any one of claims 133-140, wherein the first solution further comprises a second load component, e.g., a second nucleic acid.

142. A method of making a preparation comprising a plurality of lipid nanoparticles (LNPs), wherein the preparation is a preparation of any one of claims 115-132.

143. A method of altering a target nucleic acid, comprising:
(a) providing an LNP or preparation comprising a plurality of LNPs described herein, e.g., a LNP of any of claims 1-114, a preparation of any of claims 115-132, or a LNP or preparation made by a method of any of claims 133-142; and
(b) providing a target nucleic acid under conditions sufficient to alter the target nucleic acid,
thereby altering a target nucleic acid.

144. The method of claim 143, wherein the method is performed in an in vitro cell free system.

145. The method of claim 143, wherein the method is performed on a cell.

146. The method of claim 145, wherein the cell is a cultured cell, e.g., a cell from a cell line.

147. The method of claim 143, wherein the method is performed on a subject.

148. The method of any of claims 143-147, wherein altering comprises altering the state of association of the two strands of a target double stranded nucleic acid.

149. The method of any of claims 143-148, wherein altering comprises altering the helical structure of a target double stranded nucleic acid.

150. The method of any of claims 143-149, wherein altering comprises altering the topology, e.g., introducing a kink or bend, in a strand of target double stranded nucleic acid.

151. The method of any of claims 143-150, wherein altering comprises recruiting a nucleic acid modifying enzyme, e.g, an enzyme endogenous to a cell in which the target nucleic acid is disposed.

152. The method of any of claims 143-151, wherein altering comprises recruiting a nucleic acid modifying enzyme, e.g, a member of the nucleotide excision repair pathway, e.g., XPA, RPA, XPF, or XPG.

153. The method of any of claims 143-152, wherein altering comprises cleaving a strand of a target double stranded nucleic acid.

154. The method of any of claims 143-153, wherein altering comprises altering the sequence of the target nucleic acid.

155. The method of any of claims 143-154, wherein altering comprises altering the sequence of the target nucleic acid to the sequence of a template nucleic acid.

156. The method of any of claims 143-155, wherein altering comprises altering the sequence of the target nucleic acid to from a mutant or disorder-associated allele to a non-mutant or non-disease associated allele.

157. The method of any of claims 143-156, wherein an LNP when contacted with a target nucleic acid, allows binding of its component PNA oligomer to a target nucleic acid sequence, e.g., as evaluated by UV melting temperature in hybridization experiments, e.g., at 260 nm, thermodynamic analysis, or surface plasmon resonance.

158. The method of any of claims 143-157, wherein an LNP when contacted with a target nucleic acid, decreases the Tm of a target nucleic acid sequence, e.g., as evaluated by UV melting temperature in hybridization experiments, e.g., at 260 nm, thermodynamic analysis, or surface plasmon resonance.

159. The method of any of claims 143-158, wherein an LNP when contacted with a target nucleic acid, promotes melting or dissociation of the strands of a target nucleic acid sequence, e.g., as evaluated by a strand invasion assay.

160. The method of any of claims 143-159, wherein an LNP when contacted with a target nucleic acid, allows its component PNA oligomer to cleave a target nucleic acid sequence.

161. The method of any of claims 143-160, wherein an LNP when contacted with a target nucleic acid, allows its component PNA oligomer and nucleic acid to edit a target nucleic acid sequence, e.g., as evaluated by NGS or ddPCR.

162. A method comprising:
a) providing an LNP according to any of claims 1-114, or a preparation comprising a plurality of LNPs according to any of claims 115-132 to a cell or a subject; and
b) analyzing a sample of cells or tissue from the subject to determine if gene editing occurred in said cells or tissue.

163. The method of claim 162, wherein the contacting is performed by injection or infusion of the LNP or preparation into the bloodstream of the subject.

164. The method of claim 163, wherein the contacting is performed by injection or infusion of the LNP or preparation directly into tissue of the subject.

165. The method of any of claims 162-164, wherein the analyzing of the sample of cells or tissue is performed using digital drop polymerase chain reaction (ddPCR) or by next generation sequencing (NGS).

166. The method of claim 162-165, wherein the analysis is used to determine the percent gene editing of the cells or tissue.

167. A method comprising:
a) treating a sample of LNPs comprising a lipid and a PNA oligomer, and optionally nucleic acid(s), with a fluid comprising a detergent for a period of time suitable to depolymerize the lipid and thereby release the PNA oligomer, and optionally nucleic acid(s); and
b) analyzing the sample for the presence, absence and/or amount of the released PNA oligomer, and optionally nucleic acid(s).

168. The method of claim 167, wherein the detergent is Triton X-100.

169. The method of claim 167, further comprising making the LNP of any of claims 1-114, or preparation of LNPs of any of claims 115-132, by a method described herein.

170. A method of manufacturing, or evaluating, a LNP or preparation comprising a plurality of LNPs comprising:
b) providing a LNP or preparation comprising a plurality of LNPs described herein, e.g., a LNP of any of claims 1-114, a preparation of any of claims 115-132, or a LNP or preparation made by a method of any of claims 133-142; and
c) acquiring, directly or indirectly, a value for a preparation parameter;
thereby manufacturing, or evaluating, a LNP or preparation comprising a plurality of LNPs.

171. The method of claim 170, comprising evaluating the value for the parameter, e.g., by comparing it with a standard or reference value.

172. The method of claim 170, wherein responsive to the evaluation, selecting a course of action, and optionally, performing the action.
